(19) United States
(12) Patent Application Publication
(10) Pub. No.: US 2006/0116359 A1
(43) Pub. Date: Jun. 1, 2006

(54) 17.Beta-(alpha-hydroxy)-esters of androstanes as intermediates for the preparation of 17.beta-fluorinated-androstane esters

(76) Inventors: Gianfranco Adele Cainelli, Castelmaggiore (IT); Achille Umani Ronchi, Bologna (IT); Sergio Sandri, Forli (IT); Michele Contento, Bologna (IT); Giuseppe Fortunato, Trecate (IT); Marco Da Col, Bologna (IT); Maria Adele Boriani, Bologna (IT); Stefano Da Col, Bologna (IT)

Correspondence Address: Nixon Peabody, Clinton Square, PO Box 31051, Rochester, NY, 14603-1051, US

(21) Appl. No.: 10/538,083

(22) PCT Filed: Dec. 18, 2003
(86) PCT No.: PCT/EP03/13908

§ 371 Date: Jun. 8, 2005

(30) Foreign Application Priority Data
Dec. 9, 2002 (IT) MI2002A002606

(51) Int. Cl. A61K 31/573; C07J 3/00

(52) U.S. Cl. 514/179; 552/610

(57) ABSTRACT

Process for the preparation of polyhalogenated steroids, in particular of androstanic fluoro derivatives corticosteroids by means of the formation of new androstanic S-hydroxy alkyl or aralkyl-17-carbothioate intermediates.

### FIELD OF THE INVENTION

The present invention concerns a process for the preparation of polyhalogenated steroids, in particular of androstanic fluoro derivatives corticosteroids, and even more particularly of fluticasone propionate (S-fluoromethyl 6α,9β-difluoro-16α-methyl-3-oxo-11β-hydroxy-17α-propionyloxyandrosta-1,4-diene-17β-carbothioate) and of the intermediates thereof, by means of the formation of new androstanic S-hydroxy alkyl or aralkyl-17-carbothioate intermediates of general formula (III) herein below reported, said steroids being useful for the preparation of pharmaceutical formulations with anti-inflammatory action.

### PRIOR ART

Processes for the synthesis of polyhalogenated steroids are well known in the art, more in particular processes for the preparation of androstanic fluoro derivatives, which lead prevalently to the formation of the 6α-fluoro isomers, but all characterised by considerable difficulties such as complex purifying operations (see U.S. Pat. No. 2,961,441), the use of particularly dangerous reagents (see U.S. Pat. No. 3,980,778 and No. 4,619,921), processes with low-yield reactions and poor possibilities of industrial application (see U.S. Pat. No. 4,335,121).

The need was therefore felt to achieve new methods of synthesis of polyhalogenated steroids, in particular of androstanic fluoro derivatives corticosteroids, by means of reactions characterised by high yields, intermediates with a high degree of purity, the use of reagents easily available on the market, easily practicable and which would lead to an industrially acceptable overall yield, taking into account the number of intermediate steps.

### SUMMARY OF THE INVENTION

In the development of a new process for the preparation of polyhalogenated steroids, useful for the preparation of pharmaceutical formulations with anti-inflammatory action, the Applicant has surprisingly found a new class of androstanic S-hydroxy alkyl or aralkyl-17-carbothioate intermediates of general formula (III), as intermediates for the preparation of active drugs.

Subject of the invention are therefore compounds of general formula (III) 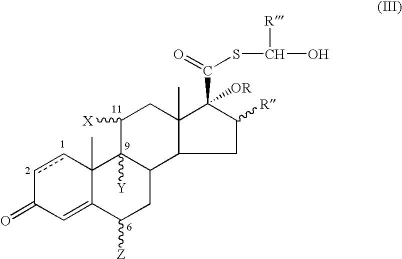
wherein
R is H or COR′ and R′ is selected from the group consisting of an alkyl group, linear or branched, having from 1 to 6 carbon atoms;
R″, in alpha or beta position with respect to the plane of the steroid reticule, is selected from the group consisting of H, an alkyl group, linear or branched, having from 1 to 5 carbon atoms; or
OR and R″, taken together, form a 16α,17α-isopropylidendioxy group or higher 16α,17α-alkylidendioxy groups, preferably having from 4 to 6 carbon atoms;
R′″ is selected from the group consisting of H, an alkyl group having from 1 to 6 carbon atoms, a phenyl or substituted phenyl group, an aralkyl or substituted aralkyl group;
X, Y and Z, in alpha or beta position with respect to the plane of the steroid reticule, equal or different from each other, are selected from the group consisting of H, OH, Cl, F, and a carbonyl group, or
X and Y, taken together, are an epoxide group or form a double bond between the positions 9 and 11; and wherein
between the positions 1 and 2 a double bond may be present.

Further subject of the invention are the processes for the preparation of the above said compounds of general formula (III) and (IV), and the process for the conversion of S-fluoromethyl 6α-fluoro-9β,11β-epoxy-16α-methyl-3-oxo-17α-propionyloxyandrosta-1,4-diene-17β-carbothioate (6) into S-fluoromethyl 6α,9α-difluoro-16α-methyl-3-oxo-11β-hydroxy-17α-propionyl oxyandrosta-1,4-diene-17β-carbothioate (18) (fluticasone propionate).

The features and advantages of the invention will be illustrated in detail in the following description.

### DETAILED DESCRIPTION OF THE INVENTION

The present compounds of general formula (III) are of particular interest as starting reagents in the synthesis of steroids useful for the preparation of pharmaceutical formulations having anti-inflammatory action.

Amongst the present compounds of general formula (III) of particular interest are the following compounds:
S-hydroxymethyl 6α-fluoro-9β,11β-epoxy-16α-methyl-3-oxo-17α-propionyloxyandrosta-1,4-diene-17β-carbothioate(5);
S-hydroxymethyl 9β,11β-epoxy-3-oxo-17α-propionyloxyandrosta-1,4-diene-17β-carbothioate (11);
S-hydroxymethyl 6α,9α-difluoro-16α-methyl-3-oxo-11β-hydroxi-17α-propionyloxyandrosta-1,4-diene-17β-carbothioate (17);
S-hydroxymethyl 6α,9α-difluoro-11β17α-dihydroxy-16α-methyl-3-oxo-androsta-1,4-diene-17β-carbothioate (21);
S-hydroxymethyl 6α,9α-difluoro-11β,16α,17α-trihydroxy-3-oxoandrosta-1,4-diene-17β-carbothioate 16,17-acetonide (27); and
S-hydroxymethyl 9β,11β-epoxy-16α,17α-dihydroxy-3-oxoandrosta-1,4-diene-17β-carbothioate 16,17-acetonide (33).

The present compounds of general formula (III), separated, identified and characterised as described herein below, are the direct precursors of androstanic S-fluoro methyl-17-carbothioates, including fluticasone propionate.

A further subject of the present invention is the process for the preparation of compounds of general formula (III) comprising the following step:
d) reaction of aldehydes of formula R′″CHO, wherein R′″ is defined as above for the compound of formula (III), said aldehydes being possibly in the form of acetal, with a compound of general formula (II) 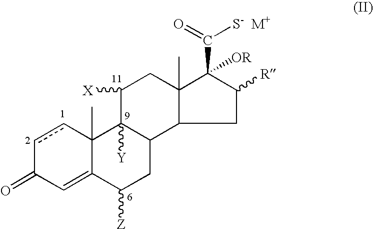
in which M⁺ is an ammonium or aminic ion, or M is H or an alkaline metal, to give a compound of general formula (III), said reaction being possibly carried out in the presence of strong mineral acids, when M is an alkaline metal or M+is an ammonium or aminic ion.

According to a preferred embodiment of the present invention in the reaction of step d) the strong mineral acid, when present, is hydrochloric acid.

According to a further preferred embodiment of the present invention in the reaction of step d) the aldehyde is formaldehyde.

The compounds of general formula (II) are easily obtainable by means of processes well known in the art such as those described in the International Patent Application No. WO 01/62722 and in the British Patent Application No. GB 2 137 206, products that can be easily isolated, identified and characterised.

Amongst embodiments of the process for the preparation of compounds of general formula (III) according to the present invention, of particular interest are the processes for the preparation of the products S-hydroxymethyl 6α-fluoro-9β,11β-epoxy-16α-methyl-3-oxo-17α-propionyloxyandrosta-1,4-diene-17β-carbothioate (5), S-hydroxymethyl 9β,11β-epoxy-3-oxo-17α-propionyloxyandrosta-1,4-diene-17β-carbothioate (11), S-hydroxymethyl 6α,9α-difluoro-16α-methyl-3-oxo-11β-hydroxy-17α-propionyloxyandrosta-1,4-diene-17β-carbothioate (17), S-hydroxymethyl 6α,9α-difluoro-11β,17α-dihydroxy-16α-methyl-3-oxo-androsta-1,4-diene-17β-carbothioate (21), S-hydroxymethyl 6α,9α-difluoro-11β,16α,17α-trihydroxy-3-oxoandrosta-1,4-diene-17β-carbothioate 16,17-acetonide (27), S-hydroxymethyl 9β,11β-epoxy-16α,17α-dihydroxy-3-oxoandrosta-1,4-diene-17β-carbothioate 16,17-acetonide (33).

A further subject of the present invention is the process for the preparation of S-hydroxymethyl 6α-fluoro-9β,11β-epoxy-16α-methyl-3-oxo-17β-propionyloxyandrosta-1,4-diene-17β-carbothioate (5) according to the general process mentioned above, in which the compound of general formula (II) is 6α-fluoro-9β,11β-epoxy-16α-methyl-17α-propionyloxy-3-oxoandrosta-1,4-diene-17β-thiocarboxylate of diethylammonium (4) which is made to react in step d) with formaldehyde to give S-hydroxymethyl 6α-fluoro-9β,11β-epoxy-16α-methyl-3-oxo-17α-propionyloxyandrosta-1,4-diene-17β-carbothioate (5).

The starting reagent, 6α-fluoro-9β,11β-epoxy-16α-methyl-17α-propionyloxy-3-oxoandrosta-1,4-diene-17β-thiocarboxylate of diethylammonium (4) can be easily prepared, upstream from the reaction in step d), by means of a process comprising the following steps:

a) reaction of 6α-fluoro-9β,11β-epoxy-17α-hydroxy-16α-methyl-3-oxoandrosta-1,4-diene-17β-carboxylic acid (1) with propionyl chloride in the presence of triethylamine to give 6α-fluoro-9β,11β-epoxy-16α-methyl-17α-propionyloxy-3-oxoandrosta-1,4-diene-17,13-carboxylic acid (2);

b) reaction of 6α-fluoro-9β,11β-epoxy-16α-methyl-17α-propionyloxy-3-oxoandrosta-1,4-diene-17β-carboxylic acid (2) coming from step a) with dimethylthiocarbamoyl chloride in the presence of sodium iodide and triethylamine to give 17β-N,N-dimethylthiocarbamoyloxycarbonyl-6α-fluoro-9β,11β-epoxy-16α-methyl-17α-propionyloxy-3-oxoandrosta-1,4-diene (3);

c) reaction of 17β-N,N-dimethylthiocarbamoiloxycarbonyl-6α-fluoro-9β,11β-epoxy-16α-methyl-17α-propionyloxy-3-oxoandrosta-1,4-diene (3) coming from step b) with diethylamine to give 6α-fluoro-9β,11β-epoxy-16α-methyl-17α-propionyloxy-3-oxoandrosta-1,4-diene-17β-thiocarboxylate of diethylammonium (4).

A further subject of the present invention is the process for the preparation of S-hydroxymethyl 9β,11β-epoxy-3-oxo-17α-propionyloxyandrosta-1,4-diene-17β-carbothioate (11) according to the general process mentioned above in which the compound of general formula (II) is 9β,11β-epoxy-17α-propionyloxy-3-oxoandrosta-1,4-diene-17β-thiocarboxylate of diethylammonium (10) which is made to react in step d) with formaldehyde to give S-hydroxymethyl 9β,11β-epoxy-3-oxo-17α-propionyloxyandrosta-1,4-diene-17β-carbothioate (11).

The starting reagent, 9β,11β-epoxy-17α-propionyloxy-3-oxoandrosta-1,4-diene-17β-thiocarboxylate of diethylammonium (10) can be easily prepared, upstream from the reaction in step d), by means of a process comprising the following steps:

a) reaction of 9β,11β-epoxy-17β-hydroxy-3-oxoandrosta-1,4-diene-17β-carboxylic acid (7) with propionyl chloride in the presence of triethylamine to give 9β,11β-epoxy-17α-propionyloxy-3-oxoandrosta-1,4-diene-17β-carboxylic acid (8);

b) reaction of 9β,11β-epoxy-17α-propionyloxy-3-oxoandrosta-1,4-diene-17β-carboxylic acid (8) coming from step a) with dimethylthiocarbamoyl chloride in the presence of sodium iodide and triethylamine to give 17β-N,N-dimethylthiocarbamoiloxycarbonyl-9β,11β-epoxy-17α-propionyloxy-3-oxoandrosta-1,4-diene (9);

c) reaction of 17β-N,N-dimethylthiocarbamoiloxycarbonyl-9β,11β-epoxy-17α-propionyloxy-3-oxoandrosta-1,4-diene (9) coming from step b) with diethylamine to give 9β,11β-epoxy-17α-propionyloxy-3-oxoandrosta-1,4-diene-17β-thiocarboxylate of diethylammonium (10).

A further subject of the present invention is the process for the preparation of S-hydroxymethyl 6α,9α-difluoro-16α-methyl-3-oxo-11β-hydroxy-17α-propionyloxyandrosta-1,4-diene-17β-carbothioate (17) according to the general process mentioned above in which the compound of general formula (II) is 6α,9α-difluoro-16α-methyl-3-oxo-11β-hydroxy-17α-propionyloxyandrosta-1,4-diene-17β-thiocarboxylate of diethylammonium (16) which is made to react in step d) with formaldehyde to give S-hydroxymethyl 6α,9α-difluoro-16α-methyl-3-oxo-11β-hydroxy-17α-propionyloxyandrosta-1,4-diene-17β-carbothioate (17).

The starting reagent, 6α,9α-difluoro-16α-methyl-3-oxo-11β-hydroxy-17α-propionyloxyandrosta-1,4-diene-17β-thiocarboxylate of diethylammonium (16) can be easily prepared, upstream from the reaction in step d), by means of a process comprising the following steps:

a) reaction of 6α,9α-difluoro-11β,17α-dihydroxy-16α-methyl-3-oxoandrosta-1,4-diene-17β-carboxylic acid (13) with propionyl chloride in the presence of triethylamine to give 6α,9α-difluoro-16α-methyl-11β-hydroxy-17α-propionyloxy-3-oxoandrosta-1,4-diene-17β-carboxylic acid (14);

b) reaction of 6α,9α-difluoro-16α-methyl-11β-hydroxy-17α-propionyloxy-3-oxoandrosta-1,4-diene-17β-carboxylic acid (14) coming from step a) with dimethylthiocarbamoyl chloride in the presence of sodium iodide and triethylamine to give 17β-N,N-dimethylthiocarbamoyloxycarbonyl-6α,9α-difluoro-16α-methyl-3-oxo-11β-hydroxy-17α-propionyloxyandrosta-1,4-diene (15);

c) reaction of 17β-N,N-dimethylthiocarbamoyloxycarbonyl-6α,9α-difluoro-16α-methyl-3-oxo-11β-hydroxy-17α-propionyloxyandrosta-1,4-diene (15) coming from step b) with diethylamine to give 6α,9α-difluoro-16α-methyl-3-oxo-11β-hydroxy-17α-propionyloxyandrosta-1,4-diene-17β-thiocarboxylate of diethylammonium (16).

A further subject of the present invention is the process for the preparation of S-hydroxymethyl 6α,9α-difluoro-16α-methyl-3-oxo-11β-hydroxy-17α-propionyloxyandrosta-1,4-diene-17β-carbothioate (17) according to the general process mentioned above in which the compound of general formula (II) is 17β carbothioic 6α,9α-difluoro-16α-methyl-3-oxo-11β-hydroxy-17α-propionyloxyandrosta-1,4-diene acid (16a) which is made to react in step d) with formaldehyde to give S-hydroxymethyl 6α,9α-difluoro-16α-methyl-3-oxo-11β-hydroxy-17α-propionyloxyandrosta-1,4-diene-17β-carbothioate (17).

The starting reagent, 17β carbothioic 6α,9α-difluoro-16α-methyl-3-oxo-11β-hydroxy-17α-propionyloxyandrosta-1,4-diene acid (16a) can be easily prepared, upstream from the reaction in step d), by means of a process comprising the following steps:

a) reaction of 6α,9α-difluoro-11β,17α-dihydroxy-16α-methyl-3-oxoandrosta-1,4-diene-17β-carboxylic acid (13) with propionyl chloride in the presence of triethylamine to give 6α,9α-difluoro-16α-methyl-11β-hydroxy-17α-propionyloxy-3-oxoandrosta-1,4-diene-17β-carboxylic acid (14);

b) reaction of 6α,9α-difluoro-16α-methyl-11β-hydroxy-17α-propionyloxy-3-oxoandrosta-1,4-diene-17β-carboxylic acid (14) coming from step a) with dimethylthiocarbamoyl chloride in the presence of sodium iodide and triethylamine to give 17β-N,N-dimethylthiocarbamoyloxycarbonyl-6α,9α-difluoro-16α-methyl-3-oxo-11β-hydroxy-17α-propionyloxy-androsta-1,4-diene (15);

c′) reaction of 17β-N,N-dimethylthiocarbamoyloxycarbonyl-6α,9α-difluoro-16α-methyl-3-oxo-11β-hydroxy-17α-propionyloxyandrosta-1,4-diene (15) coming from step b) with sodium hydrogen sulphide followed by treatment with phosphoric acid to give 17β carbothioic 6α,9α-difluoro-16α-methyl-3-oxo-11β-hydroxy-17α-propionyloxyandrosta-1,4-diene acid (16a).

A further subject of the present invention is the process for the synthesis of S-hydroxymethyl 6α,9α-difluoro-11β,17α-dihydroxy-16α-methyl-3-oxo-androsta-1,4-diene-17β-carbothioate (21) according to the general process mentioned above in which the compound of general formula (II) is 17β carbothioic 6α,9α-difluoro-11β,17α-dihydroxy-16α-methyl-3-oxo-androsta-1,4-diene acid (20) which is made to react in step d) with formaldehyde to give S-hydroxymethyl 6α,9α-difluoro-11β,17α-dihydroxy-16α-methyl-3-oxo-androsta-1,4-diene-17β-carbothioate (21). The starting reagent, 17β carbothioic 6α,9α-difluoro-11β,17α-dihydroxy-16α-methyl-3-oxo-androsta-1,4-diene acid (20) can be easily synthesised, upstream from the reaction in step d), by means of a process comprising the following steps:

b) reaction of 6α,9α-difluoro-11β,17α-dihydroxy-16α-methyl-3-oxoandrosta-1,4-diene-17β-carboxylic acid (13) with dimethylthiocarbamoyl chloride in the presence of sodium iodide and triethylamine to give 17β-N,N-dimethylthiocarbamoiloxycarbonyl-6α,9α-difluoro-11β,17α-dihydroxy-16α-methyl-3-oxoandrosta-1,4-diene (19);

c′) reaction of 17β-N,N-dimethylthiocarbamoyloxycarbonyl-6α,9α-difluoro-11β,17α-dihydroxy-16α-methyl-3-oxoandrosta-1,4-diene (19) coming from step b) with sodium hydrogen sulphide followed by treatment with phosphoric acid to give 17β carbothioic 6α,9α-difluoro-11β,17α-dihydroxy-16α-methyl-3-oxo-androsta-1,4-diene acid (20).

A further subject of the present invention is the process for the synthesis of S-hydroxymethyl 6α,9α-difluoro-11β,16α,17α-trihydroxy-3-oxoandrosta-1,4-diene-17β-carbothioate 16,17-acetonide (27) according to the general process mentioned above in which the compound with general formula (II) is 17β carbothioic 6α,9α-difluoro-11β,16α,17α-trihydroxy-3-oxoandrosta-1,4-diene 16,17-acetonide acid (26) which is made to react in step d) with formaldehyde to give S-hydroxymethyl 6α,9α-difluoro-11β,16α,17α-trihydroxy-3-oxoandrosta-1,4-diene-17β-carbothioate 16,17-acetonide (27).

The starting reagent, 17β carbothioic 6α,9α-difluoro-11β,16α,17α-trihydroxy-3-oxoandrosta-1,4-diene 16,17-acetonide acid (26) can be easily synthesised, upstream from the reaction in phase d), by means of a process comprising the following steps:

a′) alkaline hydrolysis in the presence of air of 6α,9α-difluoro-11β,16α,17α,21-tetrahydroxy-1,4-pregnadiene-3,20-dione-16,17-acetonide-21acetate (23) to give 6α,9α-difluoro-11β,16α,17α-trihydroxy-3-oxoandrosta-1,4-diene-17β-carboxylic 16,17-acetonide acid (24);

b) reaction of 6α,9α-difluoro-11β,16α,17α-trihydroxy-3-oxoandrosta-1,4-diene-17β-carboxylic 16,17-acetonide acid (24) coming from step a′) with dimethylthiocarbamoyl chloride in the presence of sodium iodide and triethylamine to give 17β-N,N-dimethylthiocarbamoyloxycarbonyl-6α,9α-difluoro-11β,16α,17α-trihydroxy-3-oxoandrosta-1,4-diene 16,17-acetonide (25);

c′) reaction of 17β-N,N-dimethylthiocarbamoiloxycarbonyl-6α,9α-difluoro-11β,16α,17α-trihydroxy-3-oxoandrosta-1,4-diene 16,17-acetonide (25) coming from step b) with sodium hydrogen sulphide followed by treatment with phosphoric acid to give 17,3 carbothioic 6α,9α-difluoro-11β,16α,17α-trihydroxy-3-oxoandrosta-1,4-diene 16,17-acetonide acid (26).

A further subject of the present invention is the process for the synthesis of S-hydroxymethyl 9β,11β-epoxy-16α,17α-dihydroxy-3-oxoandrosta-1,4-diene-17β-carbothioate 16,17-acetonide (33) according to the general process mentioned above in which the compound with general formula (II) is 17β carbothioic 9β,11β-epoxy-16α,17α-dihydroxy-3-oxoandrosta-1,4-diene 16,17-acetonide acid (32) which is made to react in step d) with formaldehyde to give S-hydroxymethyl 9β,11β-epoxy-16α,17α-dihydroxy-3-oxoandrosta-1,4-diene-17β-carbothioate 16,17-acetonide (33).

The starting reagent, 17β carbothioic 9β,11β-epoxy-16α,17α-dihydroxy-3-oxoandrosta-1,4-diene 16,17-acetonide acid (32) can be easily synthesised, upstream from the reaction in step d), by means of a process comprising the following steps:

a′) alkaline hydrolysis in the presence of air of 6α,9α-difluoro-9β,11β-epoxy-16α,17α,21-trihydroxy-1,4-pregnadiene-3,20-dione-16,17-acetonide-21acetate (29) to give 9β,11β-epoxy-16α,17α-dihydroxy-3-oxoandrosta-1,4-diene-17β-carboxylic 16,17-acetonide acid (30);

b) reaction of 9β,11β-epoxy-16α,17α-dihydroxy-3-oxoandrosta-1,4-diene-17β-carboxylic 16,17-acetonide acid (30) coming from step a′) with dimethylthiocarbamoyl chloride in the presence of sodium iodide and triethylamine to give 17β-N,N-dimethylthiocarbamoyloxycarbonyl-9β,11β-epoxy-16α,17α-dihydroxy-3-oxoandrosta-1,4-diene 16,17-acetonide (31);

c′) reaction of 17β-N,N-dimethylthiocarbamoiloxycarbonyl-9β,11β-epoxy-16α,17α-dihydroxy-3-oxoandrosta-1,4-diene 16,17-acetonide (31) coming from step b) with sodium hydrogen sulphide followed by treatment with phosphoric acid to give 17β carbothioic 9β,11β-epoxy-16α,17α-dihydroxy-3-oxoandrosta-1,4-diene 16,17-acetonide acid (32).

The compounds of general formula (III), and in particular the compounds (5), (11), (17), (21), (27) and (33), by means of a reaction of selective fluorination of the hydroxylic group in alpha position with respect to the sulphur atom, reaction in step e) after step d), are the direct precursors respectively of the compounds of general formula (IV): 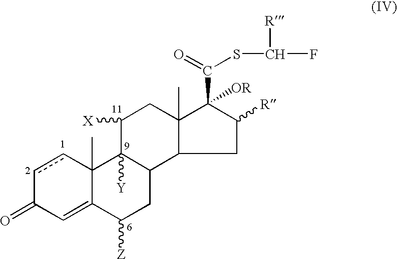
in which R, R″, R′″, X, Y and Z have the same meaning as above, of S-fluoromethyl 6α-fluoro-9β,11β-epoxy-16α-methyl-3-oxo-17α-propionyloxyandrosta-1,4-diene-17β-carbothioate (6), of S-fluoromethyl 9β,11β-epoxy-3-oxo-17α-propionyloxyandrosta-1,4-diene-17β-carbothioate (12), of S-fluoromethyl 6α,9α-difluoro-16α-methyl-3-oxo-11β-hydroxy-17α-propionyloxyandrosta-1,4-diene-17β-carbothioate (18), of S-fluoromethyl 6α,9α-difluoro-11β,17α-dihydroxy-16α-methyl-3-oxo-androsta-1,4-diene-17β-carbothioate (22), of S-fluoromethyl 6α,9α-difluoro-11β,16α,17α-trihydroxy-3-oxoandrosta-1,4-diene-17β-carbothioate 16,17-acetonide (28), and of S-fluoromethyl 9β,11β-epoxy-16α,17α-dihydroxy-3-oxoandrosta-1,4-diene-17β-carbothioate 16,17-acetonide (34).

The reaction of selective fluorination in step e) is carried out with nucleophilic fluorination reagents, preferably selected from the group consisting of bis(2-methoxyethyl) aminosulphur trifluoride (known with the trade name Deoxo-Fluor®), diethylamino sulphur trifluoride (known with the trade name DAST®), and hexafluoropropyldiethylamine (known with the trade name MEC 81®).

A further subject of the present invention is the process for the conversion of S-fluoromethyl 6α-fluoro-9β,11β-epoxy-16α-methyl-3-oxo-17α-propionyloxyandrosta-1,4-diene-17β-carbothioate (6) into S-fluoromethyl 6α,9α-difluoro-16α-methyl-3-oxo-11β-hydroxy-17α-propionyloxyandrosta-1,4-diene-17β-carbothioate (18) (fluticasone propionate) by reaction of S-fluoromethyl 6α-fluoro-9β, 11β-epoxy-16α-methyl-3-oxo-17α-propionyloxyandrosta-1,4-diene-17β-carbothioate (6) with 70% hydrofluoric acid, at a temperature ranging between −30° C. and room temperature, preferably between −20° C. and 0° C., said step f) after step e) as described above, to give S-fluoromethyl 6α,9α-difluoro-16α-methyl-3-oxo-11β-hydroxy-17α-propionyloxyandrosta-1,4-diene-17β-carbothioate (18).

The above described processes of the invention are illustrated in the following Schemes 1-8.

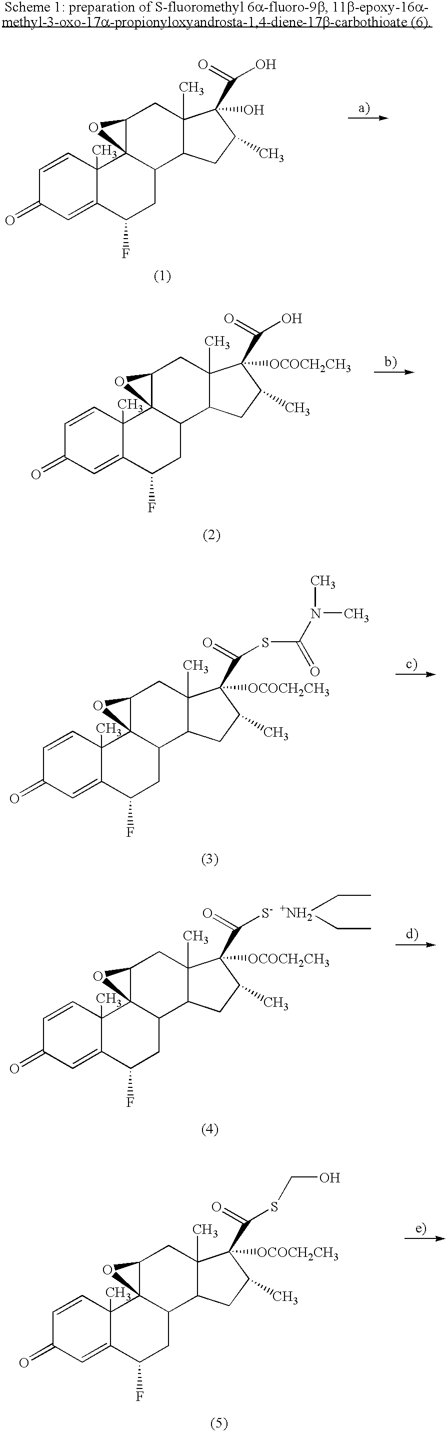

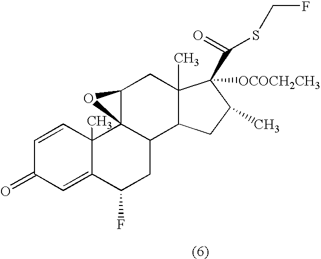

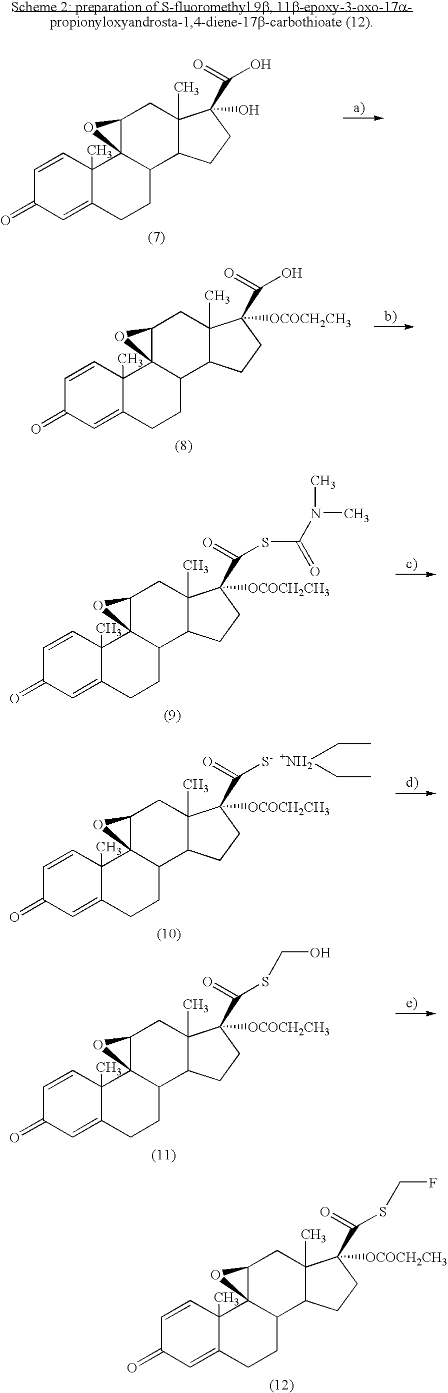

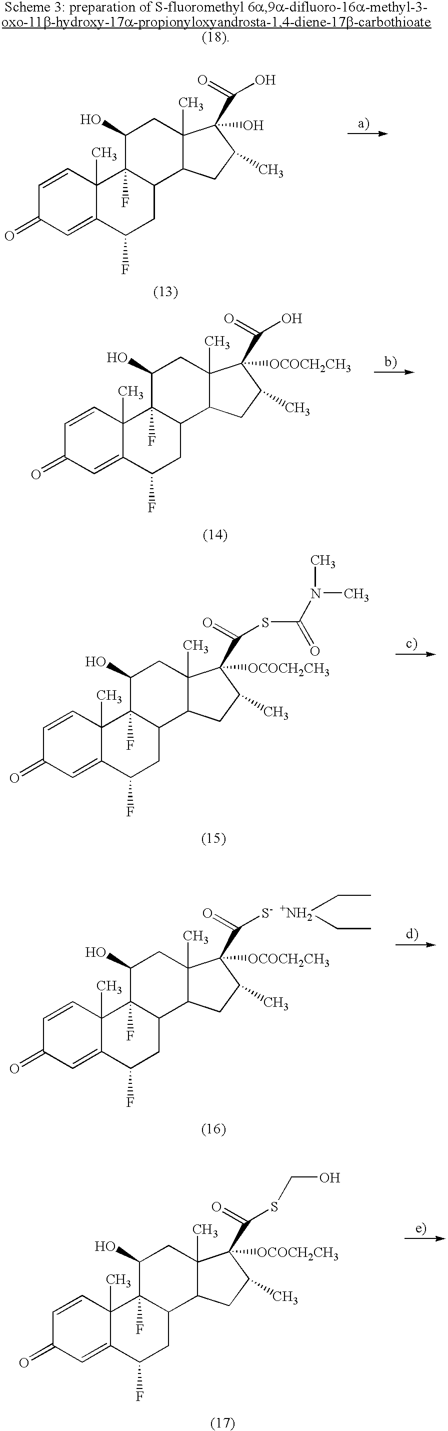

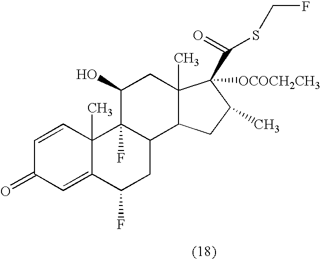

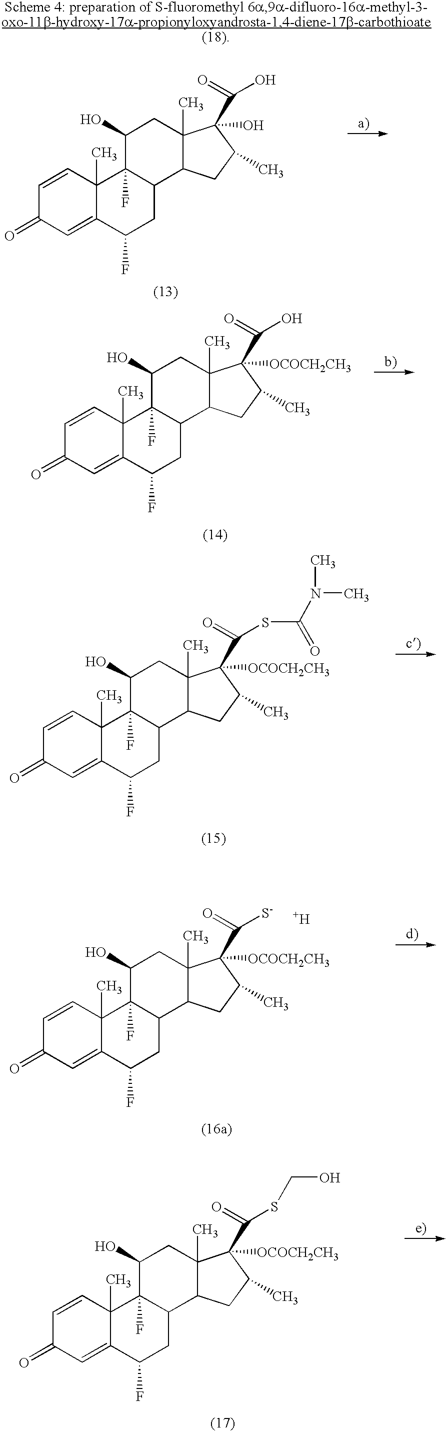

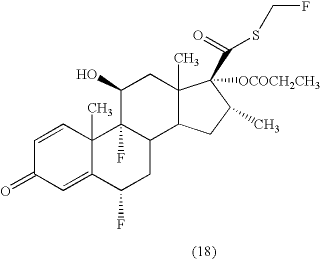

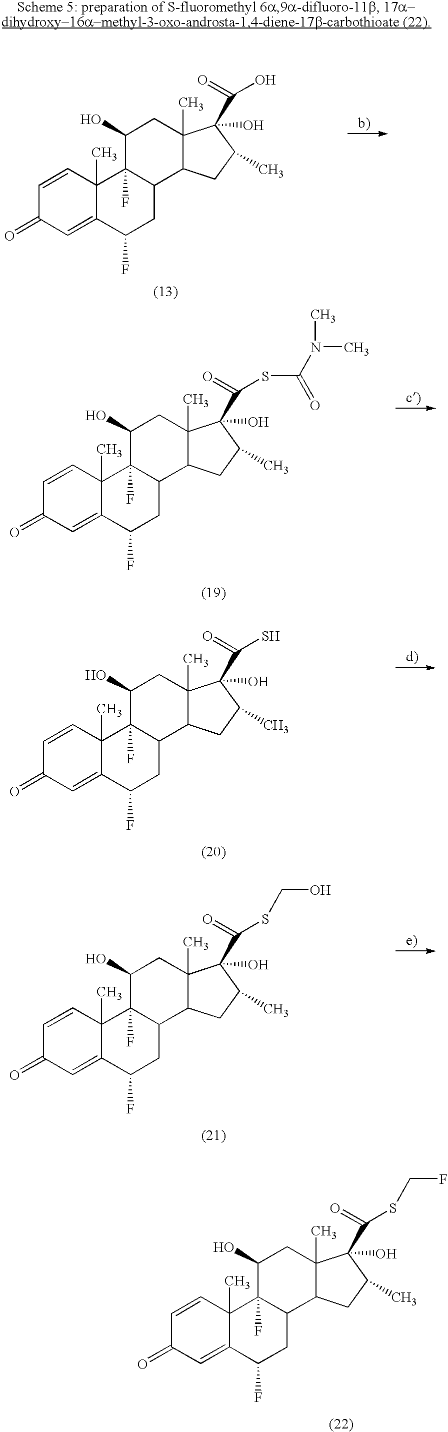

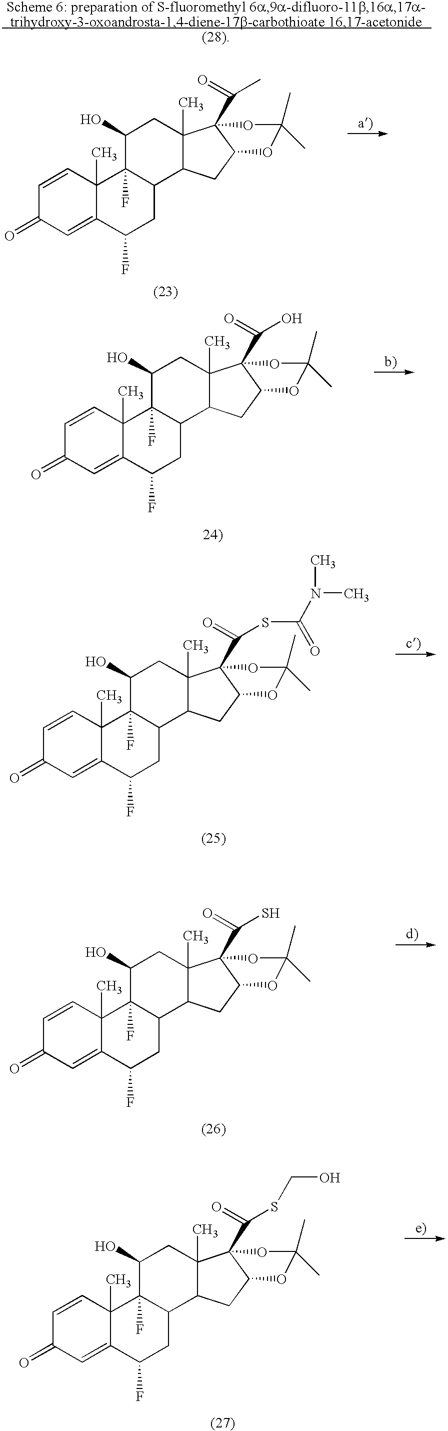

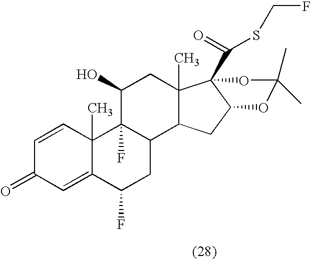

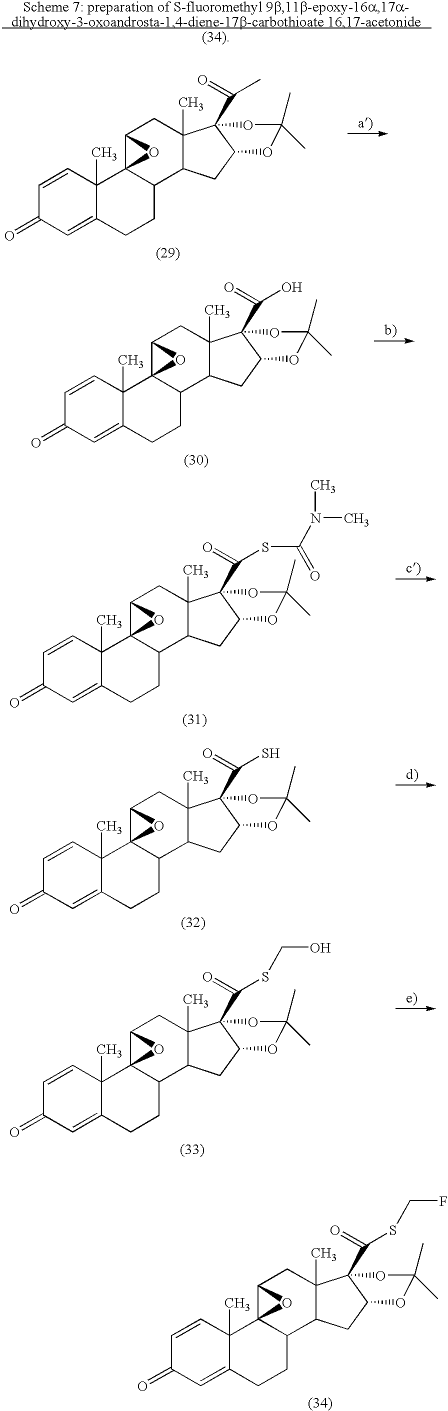

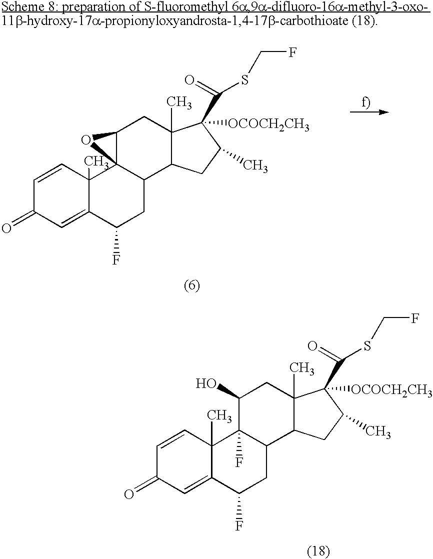

The products and the intermediates of reaction were characterised with ¹H-NMR analysis techniques.

Below are provided some examples as illustration, but without limitation, of the present invention.

### EXAMPLE 1

### Preparation of 6α-fluoro-9β,11β-epoxy-16α-methyl-17α-propionyloxy-3-oxoandrosta-1,4-diene-17β-carboxylic acid (2)

10 mmoles of 6α-fluoro-9β,11β-epoxy-17α-hydroxy-16α-methyl-3-oxoandrosta-1,4-diene-17β-carboxylic acid (1) (3.76 g) in 50 ml of CH2Cl2 and 33.5 mmoles of triethylamine (4.7 ml) are treated at 0° C. with 40 mmole of propionyl chloride (3.5 ml). The mixture is kept in agitation for about 3 hours, checking progress of the reaction with TLC (benzenes: ethyl acetate: acetic acid=6:3: 1).

On completion of the reaction the organic phase is washed 3 times with an excess of NH₄OH until pH about 10; the aqueous phases are then slowly acidified with HCl 2N until pH about 3 and the product is extracted again with CH₂Cl₂, dried on anhydrous Na₂SO₄ and finally concentrated. Yield (3.45 g): 80%.

¹H-NMR, 300 MHz: in CDCl₃; δ 0.95 (d, 3H, Me16, J=7.2 Hz); 1.06 (s, 3H, Me18); 1.18 (t, 3H, OCCH₂Me J=7.5 Hz); 1.45 (s, 3H, Me19); 2.43 (q, 2H, OCCH₂Me,J=7.5 Hz); 2.70 (m, 1H); 3.25 (m,1H); 3.34 (s,1H, H11); 5.36-5.65 (dddd, 1H, H6, J=1.5, 6.0, 11.0, 49.2 Hz); 6.31 (dd, 1H, H2, J=2.1, 10.2 Hz); 6.51 (m, 1H, H4); 6.58 (d, 1H, H1, J=10.2 Hz). The signals of the other protons fall between 1.4 and 3.2 ppm.

### EXAMPLE 2

### Preparation of 17β-N,N-dimethylthiocarbammoiloxycarbonyl-6α-fluoro-9β,11β-eoxy-16α-methyl-17α-propionyloxy-3-oxoandrosta-1,4-diene (3)

10 mmoles of 6α-fluoro-9β,11β-epoxy-16α-methyl-17α-propionyloxy-3-oxoandrosta-1,4-diene-17β-carboxylic (2) (4.32 g) in 50 ml of acetone are treated with 20 mmoles of dimethylthiocarbamoyl-chloride (2.47 g), 22 mmoles of triethylamine (3.1 ml), 1 mmole of sodium iodide (0.15 g) and finally water (0.40 ml, 10% of weight). The mixture is kept in agitation for about 3 hours at room temperature, checking the progress of the reaction with TLC (eluent: ethyl acetate). On completion of the reaction the solvent is concentrated and the residue dissolved in DMAc; this solution is dripped into cold water and the precipitate is filtered in a vacuum, washed with water and dried.

Yield (4.41 g): 85%.

¹H-NMR, 300 MHz: in CDCl₃; δ 0.98 (d, 3H, Me16, J=7.2 Hz); 1.14 (s, 3H, Me18); 1.2 (t, 3H, OCCH₂Me, J=7.5 Hz); 1.45 (s, 3H, Me19); 2.43 (q, 2H, OCCH₂Me, J=7.5 Hz); 2.70 (m 1H); 3,11 (s, 3H, NMe); 3.25 (m, 1H, 3.35 (s, 1H, H11); 5.35-5.65 (dddd, 1H, H6, J=1.5, 6.0 ,10.8, 49.2 Hz); 6.29 (dd 1H, H2, J=1.8, 9.9 Hz); 6.50 (m, 1H, H4); 6.57 (dd, 1H, H1, J=1.2, 9.9 Hz). The signals of the other protons fall between 1.5 and 3.2 ppm.

### EXAMPLE 3

### Preparation of 6α-fluoro-9β,11β-epoxy-16α-methyl-17α-propionyloxy-3-oxoandrosta-1,4-diene-17β-thiocarboxylate of diethylammonium (4)

To 10 mmoles of 17β-N,N-dimethylthiocarbammoiloxycarbonyl-6α-fluoro-9β,11β-epoxy-16α-methyl-17α-propionyloxy-3-oxoandrosta-1,4-diene (3) (5.19 g) are added 16 ml of diethylamine. The reaction mixture is heated at 60° C. (reflux temperature) and kept in agitation for 2-3 hours, checking the progress of the reaction with TLC (eluent: ethyl acetate) and solubilisation of the product. On completion of the reaction the diethylamine is concentrated and the product is obtained pure dispersing it in diethyl ether and after filtration. Yield (3.64 g): 70%.

¹H-NMR, 300 MHz: in CDCl₃; δ 0.94 (d, 3H, Me16, J=6.9 Hz); 1.07 (s, 3H, Me18); 1.14 (t, 3H, OCCH₂Me, J=7.5 Hz); 1.40(t, 6H, NCH₂Me, J=7.2 Hz); 1.45 (s, 3H, Me19); 2.41 (q, 2H, OCCH₂Me,J=7.5 Hz); 2.70 (m, 1H); 3.10 (q, 4H, NCH₂Me, J=7.2 Hz) 3.34 (s, 1H, H11); 3.60 (bs, 2H NH₂); 5.30-5.65 (dddd, 1H, H6, H=1.5, 6.0, 10.8, 49.2 Hz); 6.29 (d, 1H, H2, J=10.5 Hz); 6.49 (m, 1H, H4); 6.57 (d, 1H, H1, J=10.5 Hz). The signals of the other protons fall between 1.5 and 2.4 ppm.

### EXAMPLE 4

### Preparation of S-hydroxymethyl 6α-fluoro-9β,11β-epoxy-16α-methyl-3-oxo-17α-propionyloxyandrosta-1,4-diene-17β-carbothioate (5)

10 mmoles of 6α-fluoro-9β,11β-epoxy-16α-methyl-17α-propionyloxy-3-oxoandrosta-1,4-diene-17β-thiocarboxylate of diethylammonium (4) (5.21 gr) in 100 ml of CH₂Cl₂ are treated with 10 mmoles of HCl (2 N, 5 ml) and, after having cooled at 0° C., 50 mmoles of formalin are added (40% mN, 3.5 ml). The mixture is kept in agitation for about 30 minutes, checking the progress of the reaction with TLC (benzene: ethyl acetate: acetic acid=7:3:1). On completion of the reaction the organic phase is washed several times with slightly acid water, dried on anhydrous Na₂SO₄ and concentrated obtaining the solid product.

Yield (3.82 g): 80%.

¹H-NMR, 300 MHz: in CDCl₃; δ 0.93 (d, 3H, Me16, J=8.2 Hz); 0.98 (s, 3H, Me18); 1.12 (t, 3H, OCCH₅Me, J=7.5 Hz); 1.45 (s, 3H, Me19); 2.40 (q, 2H, OCCH₂Me, J=7.5 Hz); 2,70 (m, 1H); 3.32 (s,1H, H11); 5.10 (m, 2H, SCH₂OH); 5.25-5.62 (dddd, 1H, H6, J=1.5, 6.0, 11.0, 49.6 Hz); 6.25 (dd,1H, H2, J=1.8, 10.5 Hz); 6.44 (m, 1H, H4); 6.53 (d, 1H, H1, J=10.5 Hz). The signals of the other protons fall between 1.5 and 3.3 ppm.

### EXAMPLE 5

### Preparation of S-fluoromethyl 6α-fluoro-9β,11β-epoxy-16α-methyl-3-oxo-17α-propionyloxyandrosta-1,4-diene-17β-carbothioate (6)

To 1 mmole of S-hydroxymethyl 6α-fluoro-9β,11β-epoxy-16α-methyl-3-oxo-17α-propionyloxyandrosta-1,4-diene-17β-carbothioate (5) (0.48 g) in 10 ml of CH₂Cl₂, in an inert atmosphere and at −60° C., are slowly added 1.2 mmoles of Deoxofluor (0.22 ml). The mixture is kept in agitation for 10 minutes, checking the progress of the reaction with TLC (cyclohexane:ethyl acetate=1:1). On completion of the reaction it is washed several times with slightly alkaline water and the organic phase is concentrated and finally dried on anhydrous Na₂SO₄; the solid product is obtained with a yield of 85% (0.41 g).

¹H-NMR, 200 MHz: In CDCl₃; δ 0.94 (d, 3H, Me16, J=7.4 Hz); 1,00 (s, 3H, Me18); 1.15 (t, 3H, OCCH ₂Me, J=7.5 Hz); 1,42) (s, 3H, Me19); 2,39 (q, 2H, OCCH₂Me, J=7.5 Hz); 2.70 (m, 1H); 3.34 (s, 1H, H11); 5.25-5.63 (dddd, 1H, H6 J=1.4, 6.2, 11.0, 49.6 Hz); 5.66-6.04 (dqAB, 2H, SCH₂F, J=9.6, 50.2 Hz); 6.25 (dd, 1H, H2, J=1.4, 10.5 Hz); 6.46 (t, 1H, H4, J=1.4 Hz); 6.54 (dd, 1H, H1, J=1.4, 10.5 Hz). The signals of the other protons fall between 1.5 and 3.3 ppm.

### EXAMPLE 6

### Preparation of 9β,11β-epoxy-17α-propionyloxy-3-oxoandrosta-1,4-diene-17β-carboxylic acid (8)

10 mmoles of 9β,11β-epoxy-17α-hydroxy-3-oxoandrosta-1,4-diene-17β-carboxylic acid (7) (3.43 g) in 50 ml of CH₂Cl₂ and 33.5 mmoles of triethylamine (4.7 ml) are treated at 0° C. with 40 mmoles of propionyl chloride (3.5 ml). The mixture is kept in agitation for about 3 hours, checking the progress of the reaction with TLC (benzene: ethyl acetate: acetic acid=6:3:1).

On completion of the reaction the organic phase is washed 3 times with an excess of NH₄OH until pH about 10; the aqueous phases are then slowly acidified with HCl 2N until pH about 3 and the product is extracted again with CH₂Cl₂, dried on anhydrous Na₂SO₄ and finally concentrated. Yield (3.60 g): 90%.

¹H-NMR, 200 MHz: in CDCl₃; δ 1.00 (s; 3H, Me18); 1.15 (t, 3H, OCCH₂Me, J=7.6 Hz); 1.47 (s, 3H, Me19); 2.40 (q, 2H, OCCH₂Me, J=7.6 Hz); 3.28 (s, 1H, H11); 6.25 (m, 2H, H2, H4); 6.65 (d, 1H, H1, J=10.0 Hz). The signals of the other protons fall between 1.4 and 3.2 ppm.

### EXAMPLE 7

### Preparation of 17β-N,N-dimethylthiocarbamoiloxycarbonyl-9β,11β-epoxy-17α-propionyloxy-3-oxoandrosta-1,4-diene (9)

10 mmoles of 9β,11β-epoxy-17α-propionyloxy-3-oxoandrosta-1,4-diene-17β-carboxylic acid (8) (4.00 g) in 50 ml of acetone are treated with 20 mmoles of dimethylthiocarbamoyl-chloride (2.47 g), 22 mmoles of triethylamine (3.1 ml), 1 mmole of sodium iodide (0.15 g) and finally water (0.40 ml, 10% in weight). The mixture is kept in agitation for 3 hours at room temperature, checking the progress of the reaction with TLC (eluent: ethyl acetate). On completion of the reaction the solvent is concentrated and the residue dissolved in DMAc; this solution is dripped into cold water and the precipitate is filtered in a vacuum, washed with water and dried.

Yield (4.38 g): 90%.

¹H-NMR, 200 MHz: in CDCl₃; δ 1.00 (s, 3H, Me18); 1.19 (t, 3H, OCCH₂ME, J=7.6 Hz); 1,47 (s, 3H, Me19); 2.41 (q, 2H, OCCH₂Me, J=7.6 Hz), 3.10 (s, 6H, NMe); 3.31 (s, 1H, H11); 6.20 (m, 1H, H4); 6.24 (dd, 1H, H2, J=1.8, 10.2 Hz); 6.65 (d, 1H, H2, J=10.2 Hz). The signals of the other protons fall between 1.5 and 3.2 ppm.

### EXAMPLE 8

### Preparation of 9β,11β-epoxy-17α-propionyloxy-3-oxoandrosta-1,4-diene-17β-thiocarboxylate of diethylammonium (10)

To 10 mmoles of 17β-N,N-dimethylthiocarbamoiloxycarbonyl-9β,11β-epoxy-17α-propionyloxy-3-oxoandrosta-1,4-diene (9) (4.87 g) are added 16 ml diethylamine. The reaction mixture is heated at 60° C. (reflux temperature) and kept in agitation for about 2 hours, checking the progress of the reaction with TLC (eluent: ethyl acetate). On completion of the reaction the diethylamine is concentrated and the product is obtained pure dispersing it in diethyl ether and after filtration. Yield (2.54 g): 52%.

¹H-NMR, 300 MHz: in CDCl₃; δ 0.96 (s, 3H, Me18); 1.14 (t, 3H, OCCH₂Me, J=7.5 Hz); 1.39 (t, 6H, NCH₂Me, J=7.5 Hz); 1.48 (s, 3H, Me19) 2.43 (q, 2H, OCCH₂Me, J=7.5 Hz); 3.07 (q, 4H, NCH₂Me, J=7.5 Hz); 3.30 (s, 1H, H11); 3.50 (bs, 2H, NH₂); 6.21 (m, 1H, H4); 6.25 (dd, 1H, H2, J=1.5, 10.2 Hz); 6.67 (d, 1H, H1, J=10.2 Hz). The signals of the other protons fall between 1.2 and 3.0 ppm.

### EXAMPLE 9

### Preparation of S-hydroxymethyl 9β,11β-epoxy-3-oxo-17α-propionyloxyandrosta-1,4-diene-17β-carbothioate (11)

10 mmoles of 9β,11β-epoxy-17α-propionyloxy-3-oxoandrosta-1,4-diene-1713-thiocarboxylate of diethylammonium (10) (4.89 g) in 100 ml of CH₂Cl₂ are treated with 10 mmoles of HCl (2 N, 5 ml) and, after having cooled at 0° C., 50 mmoles of formalin are added (40% mN, 3.5 ml). The mixture is kept in agitation for about 30 minutes, checking the progress of the reaction with TLC (benzene: ethyl acetate: acetic acid=7:3:1). On completion of the reaction the organic phase is washed several times with slightly acid water, dried on anhydrous Na₂SO₄ and concentrated obtaining the solid product.

Yield (3,92 g): 80%.

¹H-NMR, 300 MHz: in CDCl₃; δ 0.98 (s, 3H, Me18); 1.16 (t, 3H, OCCH₂Me J=7.5 Hz); 1.43 (s, 3H, Me19); 2.42 (q, 2H, OCCH₂Me, J=7.5 Hz); 3.28 (t, 1H, OH, J=8.1 Hz); 3.31 (s, 1H, H11); 5.11 (dqAB, 2H, SCH₂OH, J=8.1, 10.8 Hz); 6.2 (m, 1H, H4); 6.26 (dd, 1H, H2, J=1.8, 10.2 Hz); 6.65 (d, 1H, H1, J=10.2 Hz). The signals of the other protons fall between 1.2 and 3.0 ppm.

### EXAMPLE 10

### Preparation of S-fluoromethyl 9β,11β-epoxy-3-oxo-17α-propionyloxyandrosta-1,4-diene-17β-carbothioate (12)

To 1 mmole of S-hydroxymethyl 9β,11β-epoxy-3-oxo-17α-propionyloxyandrosta-1,4-diene-17β-carbothioate (11) (0.49 gr) in 10 ml of CH₂Cl₂, in an inert atmosphere and at −60° C., are slowly added 1.2 mmoles of Deoxofluor (0.22 ml). The mixture is kept in agitation for 10 minutes, checking the progress of the reaction with TLC (cyclohexane:ethyl acetate=1:1). On completion of the reaction it is washed several times with slightly alkaline water and the organic phase is concentrated and finally dried on anhydrous Na₂SO₄; the solid product is obtained pure after chromatography on silica (cyclohexane:ethyl acetate: 20:80) with a yield of 40% (0.20 g)

¹H-NMR, 300 MHz: in CDCl₃; δ 0.94 (s, 3H, Me18); 1.20 (t, 3H, OCCH₂Me, J=7.5 Hz); 1.43 (s, 3H, Me19); 2.40 (q, 2H, OCCH₂Me, J=7.5 Hz); 3.34 (s, 1H, H11); 5.63-6.06 (dqAB, 2H, SCH₂F, J=9.4, 50.1 Hz); 6.21 (m, 1H, H4); 6.27 (dd, 1H, H2, J=1.8, 10.2 Hz); 6.65 (d, 1H, H1, J=10.2 Hz). The signals of the other protons fall between 1.3 and 3.1 ppm.

### EXAMPLE 11

### Preparation of 6α,9α-difluoro-16α-methyl-11β-hydroxy-17α-propionyloxy-3-oxoandrosta-1,4-diene-17β-carboxylic acid (14)

10 mmoles of 6α,9α-difluoro-16α-methyl-3-oxo-11β-hydroxy-17α-propionyloxyandrosta-1,4-diene-17β-carboxylic (13) (3.96 g) in 50 ml of CH₂Cl₂ and 33.5 mmoles of triethylamine (4.7 ml) are treated at 0° C. with 40 mmoles of propionyl chloride (3.5 ml). The mixture is kept in agitation for about 3 hours, checking the progress of the reaction with TLC (benzene: ethyl acetate: acetic acid=7:3:1).

On completion of the reaction the organic phase is washed 3 times with an excess of NH₄OH until pH about 10; the aqueous phases are then slowly acidified with HCl 2N until pH about 3 and the product is extracted again with CH₂Cl₂, dried on anhydrous Na₂SO₄ and finally concentrated. Yield (4.29 g): 95%.

¹H-NMR, 200 MHz: in CDCl₃; δ 0.98 (d, 3H, Me16, J=7.2 Hz); 1.17 (s, 3H, Me18); 1.20 (t, 3H, OCCH₂Me, J=7.4 Hz); 1.55 (s, 3H, Me19); 2.41 (q, 2H, OCCH₂Me, J=7.4 Hz); 3.30 (m, 1H); 4.42 (m, 1H, H11); 5.20-5.60 (dddd, 1H, H6, J=1.5, 6.2, 11.8, 49.2 Hz); 6.20 (dd, 1H, H2, J=1.8, 10.4 Hz); 6.45 (m, 1H, H4); 7.18 (d, 1H, H1, J=10.4 Hz). The signals of the other protons fall between 1.3 and 3.2 ppm.

### EXAMPLE 12

### Preparation of 17β-N,N-dimethylthiocarbamoiloxycarbonyl-6α,9α-difluoro-16α-methyl-3-oxo-11β-hydroxy-17α-propionyloxyandrosta-1,4-diene (15)

10 mmoles of 6α,9α-difluoro-16α-methyl-11β-hydroxy-17α-propionyloxy-3-oxoandrosta-1,4-diene-17β-carboxylic (14) (4.52 g) in 50 ml of acetone are treated with 20 mmoles of dimethylthiocarbamoyl-chloride (2.47 g), 22 mmoles of triethylamine (3.1 ml), 1 mmole of sodium iodide (0.15 g) and finally water (0.40 ml, 10% of weight). The mixture is kept in agitation for 3-4 hours at room temperature, checking the progress of the reaction with TLC (eluent: ethyl acetate). On completion of the reaction the solvent is concentrated and the residue dissolved in DMAc; this solution is dripped into cold water and the precipitate is filtered in a vacuum, washed with water and dried.

Yield (5,12 g): 95%.

¹H-NMR, 300 MHz: in CDCl₃; δ 1.02 (d, 3H, Me16, J=7.2 Hz); 1.18 (t, 3H, OCCH₂Me, J=7.5 Hz); 1.23 (s, 3H, Me18); 1.56 (s, 3H, Me19); 2.42 (q, 2H, OCCH₂Me,J=7.5 Hz); 3.13 (s, 3H, NMe); 3.21 (s, 3H, NMe); 3.37 (m, 1H); 5.25-5.60 (dddd, 1H, H6, J=1.5, 6.6, 11.8, 48.6 Hz); 6.41 (dd, 1H, H2, J=1.8, 10.2 Hz); 6.47 (m, 1H, H4); 7.17 (dd, 1H, H1, J=1.5, 10.2 Hz). The signals of the other protons fall between 1.3 and 2.6 ppm.

### EXAMPLE 13

### Preparation of 6α,9α-difluoro-16α-methyl-3-oxo-11β-hydroxy-17α-propionyloxyandrosta-1,4-diene-17β-thiocarboxylate of diethylammonium (16)

To 10 mmoles of 17β-N,N-dimethylthiocarbamoiloxycarbonyl-6α,9α-difluoro-16α-methyl-3-oxo-11β-hydroxy-17α-propionyloxyandrosta-1,4-diene (15) (5.39 g) are added 16 ml of diethylamine. The reaction mixture is heated at 60° C. (reflux temperature) and kept in agitation for about 2 hours, checking the progress of the reaction with TLC (eluent: ethyl acetate) and solubilisation of the product. On completion of the reaction the diethylamine is concentrated and the product is obtained pure dispersing it in diethyl ether and after filtration. Yield (3.24 g): 60%.

¹H-NMR, 300 MHz: in CDCl₃; δ 0.98 (d, 3H, Me16, J=6.9 Hz); 1.14 (t, 3H, OCCH₂Me, J=7.5 Hz); 1.19 (s, 3H, Me18); 1.43 (t, 6H, NCH₂Me, J=7.5 Hz) 1.56 (s, 3H, Me19); 2.38 (q, 2H, OCCH₂Me, J=7.5 Hz); 3.15 (q, 2H, NCH₂Me); 3.75 (bs, 2H, NH₂); 4.41 (m, 1H, H11); 5.20-5.60 (dddd,1H, H6, J=1.5, 6.6, 11.8, 49.2 Hz); 6.41 (d, 1H, H2, J=10.5 Hz); 6.47 (m, 1H, H4); 7.20 (d, 1H, H1, J=10.5 Hz). The signals of the other protons fall between 1.3 and 3.2 ppm.

### EXAMPLE 14

### Preparation of S-hydroxymethyl 6α,9α-difluoro-16α-methyl-3-oxo-11β-hydroxy-17α-propionyloxyandrosta-1,4-diene-17β-carbothioate (17)

A: 10 mmoles of 6α,9α-difluoro-16α-methyl-3-oxo-11β-hydroxy-17α-propionyloxyandrosta-1,4-diene-17β-thiocarboxylate of diethylammonium (16) (5.41 g) in 100 ml of CH₂Cl₂ are treated with 10 mmoles of HCl (2 N, 5 ml) and, after having cooled at 0° C., 50 mmoles of formalin are added (40% m/V, 3.5 ml). The mixture is kept in agitation for about an hour, checking the progress of the reaction with TLC (benzene: ethyl acetate: acetic acid=7:3:1). On completion of the reaction the organic phase is washed several times with slightly acid water, dried on anhydrous Na₂SO₄ and concentrated obtaining the solid product.

Yield (3,98 g): 80%.

B: 10 mmoles of 6α,9α-difluoro-16α-methyl-3-oxo-11β-hydroxy-17α-propionyloxyandrosta-1,4-diene-17β-thiocarboxylate of diethylammonium (16) (5.41 g) in 100 ml of DMAc are treated with 10 mmoles of HCl (2 N, 5 ml) and 10 mmole of paraformaldehyde (0.5 g). The mixture is heated at 90° C. and kept under agitation for about 3 hours, checking the progress of the reaction with TLC (benzene: ethyl acetate: acetic acid=7:3:1). On completion of the reaction the reaction mixture is dripped into cold water and the precipitate is filtered in a vacuum, washed with water and dried. Yield (2.74 g): 55%.

¹H-NMR, 200 MHz: in CDCl₃; δ 1.01 (d, 3H, Me16, J=7.0 Hz); 1.11 (s, 3H, Me18); 1.17 (t. 3H, OCCH₂Me, J=7.4 Hz); 1.54 (s, 3H, Me19); 2.38 (q, 2H, OCCH₂Me, J=7.4 Hz); 3.40 (m, 1H); 4.43 (m, 1H, H11); 5.15 (qAB, 2H, SCH₂OH, J=11.0); 5.20-5.60 (dddd. 1H, H6, J=1.5, 6.6, 11.0, 49.6 Hz); 6.43 (m, 2H, H2, H4); 7.15 (d, 1H, H1, J=10.2 Hz). The signals of the other protons fall between 1.2 and 2.7 ppm.

### EXAMPLE 15

### Preparation of S-hydroxymethyl 6α,9α-difluoro-16α-methyl-3-oxo-11β-hydroxy-17α-propionyloxyandrosta-1,4-diene-17β-carbothioate (17)

5 mmoles of 17β-N,N-dimethylthiocarbamoiloxycarbonyl-6α,9α-difluoro-16α-methyl-3-oxo-11β-hydroxy-17α-propionyloxyandrosta-1,4-diene (15) (2.70 g) are dissolved in 35 ml of DMAc and, after having cooled at 0° C., 20 mmoles of NaSH monohydrate are added (1.48 g). The mixture is kept in agitation for an hour at 0° C. and left to react for another hour at room temperature following the progress of the reaction with TLC (eluant: ethyl acetate). It is then cooled again at 0° C., water is added and H₃PO₄ diluted to pH 3 is dripped very slowly, it is extracted with 40 ml of CH₂Cl₂ and finally dried on anhydrous Na₂SO₄. The obtained solution of carbothioic 6α,9α-difluoro-16α-methyl-3-oxo-11β-hydroxy-17α-propionyloxyandrosta-1,4-diene acid (16a), cooled at 0° C., is treated with 50 mmoles of formalin (40% ml/, 3,5 ml). The mixture is kept in agitation for about an hour, checking the progress of the reaction with TLC (cyclohexane:ethyl acetate=1:1). On completion of the reaction the CH₂Cl₂ is eliminated and the remaining solution is dripped into water and ice. The precipitate obtained is filtered, washed with water and dried.

Yield (1,70 g): 75%.

¹H-NMR, 200 MHz: in CDCl₃; δ 1.01 (d, 3H, Me16, J=7.0 Hz); 1.11 (s, 3H, Me18); 1.17 (t, 3H, OCCH₂Me, J=7.4 Hz); 1,54 (s, 3H, Me19); 2.38 (q, 2H, OCCH₂Me, J=7.4 Hz); 3.40 (m, 1H); 4.43 (m, 1H, H11); 5.15 (qAB, 2H, SCH₂OH, J=11.0); 5.20-5.60 (dddd. 1H, H6, J=1.5, 6.6, 11.0, 49.6 Hz); 6.43 (m, 2H, H2, H4); 7.15 (d, 1H, H1, J=10.2 Hz). The signals of the other protons fall between 1.2 and 2.7 ppm.

### EXAMPLE 16

### Preparation of S-fluoromethyl 6α,9α-difluoro-16α-methyl-3-oxo-11β-hydroxy-17α-propionyloxyandrosta-1,4-diene-17β-carbothioate(18)

To 1 mmole of S-hydroxymethyl 6α,9α-difluoro-16α-methyl-3-oxo-11β-hydroxy-17α-propionyloxyandrosta-1,4-diene-17β-carbothioate (17) (0.50 g) in 10 ml of CH₂Cl₂, in an inert atmosphere and at −60° C., are slowly added 1.2 mmoles of Deoxofluor® (0.22 ml). The mixture is kept in agitation for 10 minutes, checking the progress of the reaction with TLC (cyclohexane:ethyl acetate=1:1). On completion of the reaction it is washed several times with slightly alkaline water and the organic phase is concentrated and finally dried on anhydrous Na₂SO₄; the solid product is obtained pure after chromatography on silica (cyclohexane:ethyl acetate=70:30) with a yield of 35% (0.17 g).

¹H-NMR, 200 MHz: in CDCl₃; δ 1.01 (d, 3H, Me16, J=7.4 Hz); 1.12 (t, 3H, OCCH₂Me, J=7.5 Hz); 1.19 (s, 3H, Me18); 1.54 (s, 3H, Me19); 2.39 (q, 2H, OCCH₂Me, J=7.5 Hz); 3,40 (m, 1H); 4.41 (m, 1H, H11); 5.25-5.60 (dddd, 1H, H6, J=1.8, 6.2, 11.0, 48.2 Hz); 5.70-6.10 (dqAB, 2H, SCH₂F, J=9.6, 50.0 Hz); 6.41 (dd, 1H, H2, J=1.8, 10.5 Hz); 6.47 (m, 1H, H4); 7.14 (dd, 1H, H1, J=1.4, 10.5 Hz). The signals of the other protons fall between 1.2 and 2.6 ppm.

### EXAMPLE 17

### Preparation of S-fluoromethyl 6α,9α-difluoro-16α-methyl-3-oxo-11β-hydroxy-17α-propionyloxyandrosta-1,4-diene-17β-carbothioate (18)

To 1 mmole of S-hydroxymethyl 6α,9α-difluoro-16α-methyl-3-oxo-11β-hydroxy-17α-propionyloxyandrosta-1,4-diene-17β-carbothioate (17) (0.50 g) in 10 ml of THF, in an inert atmosphere and at −20° C., are slowly added 1.5 mmoles of DAST® (0.20 ml). The mixture is kept in agitation for 20 minutes, checking the progress of the reaction with TLC (cyclohexane:ethyl acetate=1:1). On completion of the reaction it is diluted with ethyl acetate, washed several times with slightly alkaline water and the organic phase is dried on anhydrous Na₂SO₄ and finally concentrated; the pure solid product is obtained with a yield of 68% (0.34 g).

¹H-NMR, 200 MHz: in CDCl₃; δ 1.01 (d, 3H, Me16, J=7.4 Hz); 1.12 (t, 3H, OCCH₂Me, J=7.5 Hz); 1,19 (s, 3H, Me18); 1.54 (s, 3H, Me19); 2.39 (q, 2H, OCCH₂Me, J=7.5 Hz); 3.40 (m, 1H); 4.41 (m, 1H, H11); 5.25-5.60 (dddd, 1H, H6, J=1.8, 6.2, 11.0, 48.2 Hz); 5.70-6.10 (dqAB, 2H, SCH₂F, J=9.6, 50.0 Hz); 6.41 (dd, 1H, H2, J=1.8, 10.5 Hz); 6.47 (m, 1H, H4); 7.14 (dd, 1H, H1, J=1.4, 10.5 Hz). The signals of the other protons fall between 1.2 and 2.6 ppm.

### EXAMPLE 18

### Preparation of 17β-N,N-dimethylthiocarbamoiloxycarbonyl-6α,9α-difluoro-11β,17α-dihydroxy-16α-methyl-3-oxoandrosta-1,4-diene (19)

10 mmoles of 6α,9α-difluoro-11β,17α-dihydroxy-16α-methyl-3-oxoandrosta-1,4-diene-17β-carboxylic (13) (3.98 g) in 50 ml of acetone are treated with 20 mmoles of dimethylthiocarbamoylchloride (2.47 g), 22 mmoles of triethylamine (3.1 ml), 1 mmole of sodium iodide (0.15 g) and finally water (0.40 ml, 10% of weight). The mixture is kept in agitation for 3-4 hours at room temperature, checking the progress of the reaction with TLC (eluent: ethyl acetate). On completion of the reaction the solvent is concentrated and the residue dissolved in DMAc; this solution is dripped into cold water to cause precipitation of the product which is then filtered and dried. Yield: 96% (4.64 g).

¹H-NMR, 200 MHz: in CDCl₃; δ 1.02 (d, 3H, Me16, J=7.2 Hz); 1.17 (s, 3H, Me18); 1.56 (s, 3H. Me19); 3.12 (s, 3H, NMe); 3.16 (s, 3H, NMe); 4.43 (m, 1H, H11); 5.20-5.60 (dddd, 1H, H6, J=1.5, 6.6, 11.8, 48.6 Hz); 6.40 (dd, 1H, H2, J=1.8, 10.2 Hz); 6.46 (m, 1H, H4); 7.15 (dd, 1H, H1, J=1.4, 10.2 Hz). The signals of the other protons fall between 1.3 and 2.6 ppm.

### EXAMPLE 19

### Preparation of 17β carbothioic 6α,9α-difluoro-11β,17α-dihydroxy-16α-methyl-3-oxo-androsta-1,4-diene acid (20)

5 mmoles of 17β-N,N-dimethylthiocarbamoiloxycarbonyl-6α,9α-difluoro-11β,17α-dihydroxy-16α-methyl-3-oxoandrosta-1,4-diene (19) (2.42 g) dissolved in 40 ml of DMAc and cooled at 0° C., are treated with 20 mmoles of NaSH Monohydrate 1.48 g). The reaction mixture is kept in agitation for an hour at 0° C. and another hour at room temperature checking the progress of the reaction with TLC (eluent: ethyl acetate). On completion of the reaction the reaction mixture is dripped into slightly acid cold water maintaining a pH of about 3; the product precipitates as a white solid. After filtration the product was dissolved in 70 ml of CH₂Cl₂ for the subsequent reaction.

¹H-NMR, 200 MHz; in CDCl₃, δ 0.99 (d, 3H, Me16, J=7.2 Hz); 1.12 (s, 3H, Me18); 1.55 (s, 3H, Me19); 3.09 (m, 1H); 4.43 (M, 1H, H11); 4.68 (bs, 1H, SH); 5.20-5.60 (dddd, 1H, H6, J=1.5, 6.6, 11.849.2 Hz); 6.39 (dd, 1H, H2, J=1.8, 10.0 Hz); 6.45 (s, 1H, H4); 7.13 (dd, 1H, H1, J=1.5, 9.6 Hz): The signals of the other protons fall between 1.3 and 3.2 ppm.

### EXAMPLE 20

### Preparation of S-hydroxymethyl 6α,9α-difluoro-11β,17α-dihydroxy-16α-methyl-3-oxo-androsta-1,4-diene-17β-carbothioate (21)

5 mmoles (theoretical) of 170 carbothioic 6α,9α-difluoro-1β,17α-dihydroxy-16α-methyl-3-oxo-androsta-1,4-diene acid (20) in 70 ml of CH₂Cl₂ cooled at 0° C., are treated with 20 mmoles of formalin (40% m/V, 1.38 ml). The mixture is kept in agitation for about an hour, checking the progress of the reaction with TLC (eluent: ethyl acetate). On completion of the reaction the reaction product is extracted with CH₂Cl₂; the organic phase was dried on anhydrous Na₂SO₄ and concentrated. The solid product was obtained with a yield of 65% (1.44 g).

¹H-NMR, 200 MHz: in CDCl₃; δ 0.99 (d, 3H, Me16, J=7.2 Hz); 1.10 (s, 3H, Me18); 1.54 (s, 3H, Me19); 3.11 (m, 1H); 4.39 (m, 1H, H11); 5.06 (m, 2H, SCH₂HO); 5.15-5.60 (dddd, 1H, H6, J=1.5, 6.6 11.049.6 Hz); 6.39 (dd, 1H, H2, J=1.8, 10.0 Hz); 6.44 (m, 1H, H4); 7.13 (dd, 1H, H1, J=1.5, 10.0 Hz). The signals of the other protons fall between 1.2 and 2.7 ppm.

### EXAMPLE 21

### Preparation of S-fluoromethyl 6α,9α-difluoro-11β,17α-dihydroxy-16α-methyl-3-oxo-androsta-1,4-diene-17β-carbothioate (22)

To 1 mmole of S-hydroxymethyl 6α,9α-difluoro-11β,17α-dihydroxy-16α-methyl-3-oxo-androsta-1,4-diene-17β-carbothioate (21) (0.44 g) in 10 ml of CH₂Cl₂ in an inert atmosphere and at 60° C., are slowly added 1.2 mmoles of Deoxofluor (0.22 ml). The mixture is kept in agitation for 10 minutes, checking the progress of the reaction with TLC (cyclohexane:ethyl acetate=1:1). On completion of the reaction it is washed several times with slightly alkaline water and the organic phase is dried on anhydrous Na₂SO₄ and finally concentrated; the pure solid product is obtained by chromatography on silica (cyclohexane:ethyl acetate=60:40) with a yield of 35% (0.16 g).

¹H-NMR, 200 MHz: in CDCl₂; δ 1.00 (d, 3H, Me16, J=7.4 Hz); 1.10 (s, 3H, Me18); 1.52 (s, 3H, Me19); 3.15 (m, 1H); 4.40 (m, 1H, H11); 5.10-5.60 (dddd, 1H, H6, J=1.8, 6.2 11.048.2 Hz); 5.86 (dqAB, 2H, SCH₂F, J=9.6, 50.0 Hz); 6.38 (dd, 1H, H2, J=1.8, 10.2 Hz); 6.44 (m, 1H, H4); 7.14 (dd, 1H, H1, J=1.4, 10.2 Hz). The signals of the other protons fall between 1.2 and 2.6 ppm.

### EXAMPLE 22

### Preparation of 6α,9α-difluoro-11β,16α,17α-trihydroxy-3-oxoandrosta-1,4-diene-17β-carboxylic 16,17-acetonide acid (24)

To 7.00 g of 6α,9α-difluoro-11β,16α,17α,21-tetrahydroxy-1,4-pregnadiene-3,20-dione-16,17-acetonide-21acetate (23) (14.17 mmoles) are added 90 ml of EtOH and, after having cooled at 0° C., 3.17 g of KOH (4 equivalent) dissolved in 30 ml of EtOH are dripped. The reaction mixture was left under the air flow by tempering for 2.5 hours, during which time the progress of the reaction was checked with TLC (cyclohexane: EtOAc=1:3). On completion of the reaction the solvent was concentrated, the residue dissolved again in water and washed with EtOAc (twice); the aqueous phase was acidified with H₃PO₄ diluted to pH=3, and extracted with EtOAc, dried with anhydrous Na₂SO₄ and finally concentrated. The product 6α,9α-difluoro-11β,16α,17α-trihydroxy-3-oxoandrosta-1,4-diene-17β-carboxylic 16,17-acetonide acid (24) was obtained as a white solid with a yield of 89% (5.50 g).

¹H-NMR, 200 MHz: in CDCl₃; δ 1.00 (s, 3H, Me18); 1.25 (s, 3H, Me); 1.39 (s, 3H, Me); 1.51 (s, 3H, Me); 4.27 (m, 1H, H11); 5.09(d, 1H, H16, J=3.0 Hz); 5.10-5.50 (dddd, 1H, H6, J=1.5, 6.2 11.849.2 Hz); 6.33 (dd, 1H, H2, J=1.810.0 Hz); 6.39 (m, 1H, H4); 7.18 (dd, 1H, H1, J=1.4, 10.0 Hz). The signals of the other protons fall between 1.3 and 3.2 ppm.

### EXAMPLE 23

### Preparation of the product 17β-N,N-dimethylthiocarbamoiloxycarbonyl-6α,9α-difluoro-11β,16α,17α-trihydroxy-3-oxoandrosta-1,4-diene 16,17-acetonide (25)

10 mmoles of 6α,9α-difluoro-11β,16α,17α-trihydroxy-3-oxoandrosta-1,4-diene-17β-carboxylic 16,17-acetonide acid (24) (4.38 g) in 50 ml of acetone are treated with 20 mmoles of dimethylthiocarbamoilchloride (2.47 g), 22 mmoles of triethylamine (3.1 ml), 1 mmole of sodium iodide (0.15 g) and finally water (0.40 ml, 10% of weight). The mixture is kept in agitation for 3-4 hours at room temperature, checking the progress of the reaction with TLC (eluent: ethyl acetate). On completion of the reaction the solvent is concentrated and the residue dissolved in AcOEt, washed with slightly alkaline water. The organic phase is dried on anhydrous Na₂SO₄ and concentrated. The solid product is obtained with a yield of 50% (2.62 g).

¹H-NMR, 200 MHz: in CDCl₂; δ 1.07 (s, 3H, Me18); 1.32 (s, 3H, Me); 1.47 (s, 3H, Me); 1.55 (s, 3H, Me); 3.11 (s, 3H, NMe); 4.49 (m, 1H, H11, 5.05 (d, 1H, H16, J=3.8 Hz); 5.10-5.60 (dddd, 1H, H6, J=1.5, 6.6 11.848.6 Hz); 6.39 (dd, 1H, H2, J=1.8, 10.2 Hz); 6.45 (m, 1H, H4); 7.19 (dd, 1H, H1, J=1.4, 10.2 Hz). The signals of the other protons fall between 1.3 and 2.6 ppm.

### EXAMPLE 24

### Preparation of 17β carbothioic 6α,9α-difluoro-11β,16α,17α-trihydroxy-3-oxoandrosta-1,4-diene 16,17-acetonide acid (26)

5 mmoles of 17β-N,N-dimethylthiocarbamoiloxycarbonyl-6α,9α-difluoro-11β,16α,17α-trihydroxy-3-oxoandrosta-1,4-diene 16,17-acetonide (25) (2.62 g) dissolved in 40 ml of DMAc and cooled at 0° C., are treated with 20 mmoles of NaSH monohydrate (1.48 g). The reaction mixture is kept in agitation for an hour at 0° C. and another hour at room temperature checking the progress of the reaction with TLC (eluant: ethyl acetate). On completion of the reaction the reaction mixture is dripped into slightly acid cold water; the product precipitates as a yellow solid maintaining a pH of about 3. After filtration the product was dissolved in 70 ml of CH₂Cl₂ for the subsequent reaction.

¹H-NMR, 300 MHz: in CDCl₂; δ 1.05 (s, 3H, Me18); 1.34 (s, 3H, Me); 1.49 (s, 3H, Me); 1.57 (s, 3H, Me); 4.46 (m, 1H, H11); 4.91 (bs, 1H, SH); 5.01 (d. 1H, H16, J=5.4 Hz); 5.20-5.60 (dddd, 1H, H6, J=1.5, 6.6 11.849.2 Hz); 6.42 (dd, 1H, H2, J=1.8, 9.6 Hz); 6.48 (s, 1H, H4); 7.15 (dd, 1H, H1, J=1.5, 9.6 Hz). The signals of the other protons fall between 1.3 and 3.2 ppm.

### EXAMPLE 25

### Preparation of S-hydroxymethyl 6α,9α-difluoro-11β,16α,17α-trihydroxy-3-oxoandrosta-1,4-diene-17β-carbothioate16,17-acetonide (27)

5 mmoles (theoretical) of 17β carbothioic 6α,9α-difluoro-11β,16α,17α-trihydroxy-3-oxoandrosta-1,4-diene 16,17-acetonide acid (26) in 70 ml of CH₂CL₂ cooled at 0° C., are treated with 20 mmoles of formalin (40% mN, 1.38 ml). The mixture is kept in agitation for about an hour, checking the progress of the reaction with TLC (eluent: ethyl acetate) and observing precipitation of the product as a white solid. On completion of the reaction the product was filtered and obtained with a yield of 62% (1.50 g).

¹H-NMR, 200 MHz: in CDCl₃; δ 0.92 (s, 3H, Me18); 1.20 (s, 3H, Me); 1.40 (s, 3H, Me); 1.50 (s, 3H, Me); 4.28 (m, 1H, H11); 4.87(d, 2H, SCH₂OH, J=11.0); 4.99 (d, 1H, H16, J=34); 5.21 (d, 1H, SCH₂OH, J=11.0); 5.20-5.60 (dddd, 1H, H6, J=1.5, 6,6, 11.0, 49.6 Hz); 6.34 (dd, 1H, H2, J=1.8, 10.2 Hz); 6.39 (m, 1H, H4); 7.17 (dd, 1H, H1, J=1.5, 10.2 Hz). The signals of the other protons fall between 1.2 and 2.7 ppm.

### EXAMPLE 26

### Preparation of S-fluoromethyl 6α,9α-difluoro-11β,16α,17α-trihydroxy-3-oxoandrosta-1,4-diene-17β-carbothioate16,17-acetonide (28)

To 1 mmole of S-hydroxymethyl 6α,9α-difluoro-11β,16α,17α-trihydroxy-3-oxoandrosta-1,4-diene-17β-carbothioate16,17-acetonide (27) (0.48 g) in 10 ml of CH₂Cl₂ in an inert atmosphere and at −15° C., are slowly added 1.0 mmoles of DAST (0.13 ml). The mixture is kept in agitation for an hour, checking the progress of the reaction with TLC (cyclohexane ethyl acetate=1:1). On completion of the reaction it is washed several times with slightly alkaline water and the organic phase is dried on anhydrous Na₂SO₄ and finally concentrated; the pure solid product is obtained by chromatography on silica (cyclohexane:ethyl acetate=80:20) with a yield of 30% (0.15 g).

¹H-NMR, 200 MHz: in CDCl₃; δ 1.01 (s, 3H, Me18); 1.23 (s, 3H, Me); 1.45 (s, 3H, Me); 1.65 (s, 3H, Me); 4.46 (m, 1H, H11); 5.03 (d, 1H, H16, J=4.0 Hz); 5.20-5.70 (dddd, 1H, H6, J=1.8, 6.2, 11.0, 49.2 Hz); 5.86 (dqAB, 2H, SCH₂F, J=9.6, 50.6 Hz); 6.25 (m, 1H, H4); 6.29 (dd, 1H, H2, J=1.8, 10.0 Hz); 7.30 (dd, 1H, H1, J=1.6, 10.0 Hz). The signals of the other protons fall between 1.2 and 2.6 ppm.

### EXAMPLE 27

### Preparation of 9β,11β-epoxy-16α,17α-dihydroxy-3-oxoandrosta-1,4-diene-17β-carboxylic 16,17-acetonide acid (30)

To 10.00 gr of 6α,9α-difluoro-9β,11β-epoxy-16α,17 α,21-trhydroxy-1,4-pregnadiene-3,20-dione-16,17-acetonide-21acetate (29) (21.93 mmoles) are added 130 ml of EtOH and 4.91 g of KOH (4 equivalent; 87.72 mmoles) dissolved in 70 ml of EtOH are dripped. The reaction mixture was left under the air flow for 2.5 hours, during which time the progress of the reaction was checked with TLC (cyclohexane: EtOAc=1:3). On completion of the reaction the solvent was concentrated, the residue dissolved again in water and washed with EtOAc (twice); the aqueous phase was acidified with H₃PO₄ diluted to pH=3, extracted with EtOAc, dried with anhydrous Na₂SO₄ and finally concentrated. The product 9β,11β-epoxy-16α,17α-dihydroxy-3-oxoandrosta-1,4-diene-17β-carboxylic 16,17-acetonide acid (30) was obtained pure after reprecipitation from Et₂O/EtOAc as a white solid with a yield of 84% (7.37 g).

¹H-NMR, 200 MHz: in CDCl₃; δ 1.00 (s, 3H, Me18); 1.30 (s, 3H, Me); 1.46 (s, 3H, Me); 1.47 (s, 3H, Me), 3.29 (s, 1H, H11); 5.10 (d, 1H, H16, J=4.6 Hz); 6.22 (m, 1H, H4); 6.27 (dd, 1H, H2, J=1.8, 10.2 Hz); 6.65 (dd, 1H, H1, J=1.4, 10.2 Hz) The signals of the other protons fall between 1.0 and 2.8 ppm.

### EXAMPLE 28

### Preparation of 17β-N,N-dimethylthiocarbamoiloxycarbonyl-9β,11β-epoxy-16α,17α-dihydroxy-3-oxoandrosta-1,4-diene 16,17-acetonide (31)

10 mmoles of 9β,11β-epoxy-16α,17α-dihydroxy-3-oxoandrosta-1,4-diene-17β-carboxylic 16,17-acetonide acid (30) (4.00 g) in 50 ml of acetone are treated with 20 mmoles of dimethylthiocarbamoilchloride (2.47 g), 22 mmoles of triethylamine (3.1 ml), 1 mmole of sodium iodide (0.15 g) and finally water (0.40 ml, 10% of weight). The mixture is kept in agitation for 3-4 hours at room temperature, checking the progress of the reaction with TLC (eluent: ethyl acetate). On completion of the reaction the solvent is concentrated and the residue dissolved in AcOEt, washed with slightly alkaline water. The organic phase is dried on anhydrous Na₂SO₄ and concentrated. The solid product is obtained with a yield of 50% (2.44 g).

¹H-NMR, 200 MHz: in CDCl₃; δ 1.00 (s, 3H, Me18); 1.32 (s, 3H, Me); 1.45 (s, 3H, Me); 1.48 (s, 3H, Me); 3.09 (s, 3H, NMe); 3.13 (s, 3H, NMe); 3.28 (s, 1H, H11); 4.99 (d, 1H, H16, J=4.8, Hz); 6.19 (m, 1H, H4); 6.24 (dd, 1H, H2, J=1.8, 10.0 Hz); 6.63 (dd, 1H, H1, J=1.4, 10.0 Hz). The signals of the other protons fall between 1.2 and 2.8 ppm.

### EXAMPLE 29

### Preparation of 17β carbothioic 9β,11β-epoxy-16α,17α-dihydroxy-3-oxoandrosta-1,4-diene 16,17-acetonide acid (32)

5 mmoles of 17β-N,N-dimethylthiocarbamoiloxycarbonyl-9β,11β-epoxy-16α,17α-dihydroxy-3-oxoandrosta-1,4-diene 16,17-acetonide (31) (2.44 g) dissolved in 40 ml of DMAc and cooled at 0° C., are treated with 20 mmoles of NaSH monohydrate (1.48 g). The reaction mixture is kept in agitation for an hour at 0° C. and another hour at room temperature checking the progress of the reaction with TLC (eluent: ethyl acetate). On completion of the reaction the reaction mixture is dripped into slightly acid cold water; the product precipitates as a yellow solid maintaining a pH of about 3. After filtration the product was dissolved in 70 ml of CH₂Cl₂ for the subsequent reaction.

¹H-NMR, 300 MHz: in CDCl₃; δ 1.00 (s, 3H, Me18); 1.33 (s, 3H, Me); 1.46 (s, 3H, Me); 1.48 (s, 3H, Me); 3.32 (s, 1H, H11); 4.81 (bs, 1H, SH); 4.97 (d, 1H, H16, J=5.1, Hz); 6.22 (m, 1H, H4); 6.27 (dd, 1H, H2, J=1.8, 10.2 Hz); 6.65 (dd, 1H, H1, J=1.4, 10.2 Hz). The signals of the other protons fall between 1.0 and 3.0 ppm.

### EXAMPLE 30

### Preparation of S-hydroxymethyl 9β,11β-epoxy-16α,17α-dihydroxy-3-oxoandrosta-1,4-diene-17β-carbothioate16,17-acetonide (33)

5 mmoles (theoretical) of 17β carbothioic 9β,11β-epoxy-16α,17α-dihydroxy-3-oxoandrosta-1,4-diene 16,17-acetonide acid (32) in 70 ml of CH₂Cl₂, cooled at 0° C., are treated with 20 mmoles of formalin (40% m/V, 1.38 ml). The mixture is kept in agitation for about three hours, checking the progress of the reaction with TLC (eluent, ethyl acetate). The reaction was not completed and the extracted product was obtained non pure with a yield of 92% (2.07 g) and it was used just as it was in the subsequent reaction.

¹H-NMR, 300 MHz: in CDCl₃; δ 0.92 (s, 3H, Me18); 1.28 (s, 3H, Me); 1.47 (s, 3H, Me); 1.50 (s, 3H, Me); 3.30 (s, 1H, H11); 5.02 (d, 1H, H16, J=4.8 Hz); 5.11 (qAB, 2H, SCH₂OH, J=11.0) 6.21 (m, 1H, H4); 6.26 (dd, 1H, H2, J=1.8, 9.9 Hz); 6.63 (dd, 1H, H1, J=1.4, 9.9 Hz). The signals of the other protons fall between 1.0 and 2.9 ppm.

### EXAMPLE 31

### Preparation of S-fluoromethyl 9β,11β-epoxy-16α,17α-dihydroxy-3-oxoandrosta-1,4-diene-17β-carbothioate16,17-acetonide (34)

To 1 mmole of S-hydroxymethyl 9β,11β-epoxy-16α,17α-dihydroxy-3-oxoandrosta-1,4-diene-17β-carbothioate16,17-acetonide (33) (0.45 g) in 10 ml of CH₂Cl₂ in an inert atmosphere and at −15° C., are slowly added 1.0 mmoles of DAST (0.13 ml). The mixture is kept in agitation for two hours, checking the progress of the reaction with TLC (cyclohexane:ethyl acetate=1:1). On completion of the reaction it is washed several times with slightly alkaline water and the organic phase is dried on anhydrous Na₂SO₄ and finally concentrated; the pure solid product is obtained by chromatography on silica (cyclohexane:ethyl acetate=90:10) with a yield of 40% (0.18 g).

¹H-NMR, 200 MHz: in CDCl₃; δ 0.90 (s, 3H, Me18); 1.24 (s, 3H, Me); 1.45 (s, 3H, Me); 1.49 (s, 3H, Me); 1.49 (s, 3H, Me); 3.29 (s,1H, H11); 5.01 (d, 1H, H16, J=4.8 Hz); 5.75-6.10 (dqAB, 2H, SCH₂F, J=9.2, 50.0 Hz); 6.20 (m, 1H, H4); 6.24 (dd, 1H, H2, J=1.8, 10.0 Hz); 6.62 (dd, 1H, H1, J=1.4, 10.0 Hz). The signals of the other protons fall between 1.0 and 2.8 ppm.

### EXAMPLE 32

### Preparation of Fluticasone Propionate

To 4 ml of hydrofluoric acid 70%, cooled at −20° C., are added in portions 0.850 g. of S-fluoromethyl 6α-fluoro-9β,11β-epoxy-16α-methyl-3-oxo-17α-propionyloxyandrosta-1,4-diene-17β-carbothioate (6), prepared according to Example 5, and the mixture is left to react at the same temperature for 7 hours, then poured slowly into diluted ammonia (50 ml) and the suspension obtained is neutralised at pH=8.5.

The solid is filtered, washed with water until neutrality and dried. 0.620 g of raw S-fluoromethyl 6α,9α-difluoro-16α-methyl-3-oxo-11β-hydroxy-17α-propionyloxyandrosta-1,4-diene-17β-carbothioate (18) (fluticasone propionate) are obtained, the identity of which is demonstrated by comparison with an authentic sample prepared according to the International Patent Application No. WO 01/62722.

1. Compounds of general formula (III): 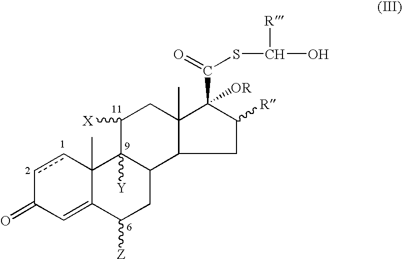
wherein R is H or COR′ and R′ is selected from the group consisting of an alkyl group, linear or branched, having from 1 to 6 carbon atoms;
R″, in alpha or beta position with respect to the plane of the steroid reticule, is selected from the group consisting of H, an alkyl group, linear or branched, having from 1 to 5 carbon atoms; or
OR and R″, taken together, form a 16α,17α-isopropylidendioxy group or higher 16α,17α-alkylidendioxy groups, preferably having from 4 to 6 carbon atoms;
R′″ is selected from the group consisting of H, an alkyl group having from 1 to 6 carbon atoms, a phenyl or substituted phenyl group, an aralkyl or substituted aralkyl group;
X, Y and Z, in alpha or beta position with respect to the plane of the steroid reticule, equal or different from each other, are selected from the group consisting of H, OH, Cl, F, and a carbonyl group, or
X and Y, taken together, are an epoxide group or form a double bond between the positions 9 and 11;
and wherein between the positions 1 and 2 a double bond may be present.

2. Compounds according to claim 1, selected from the group consisting of:
S-hydroxymethyl 6 α-fluoro-9β,11β-epoxy-16α-methyl-3-oxo-17α-propionyloxyandrosta-1,4-diene-17β-carbothioate (5);
S-hydroxymethyl 9β,11β-epoxy-3-oxo-17α-propionyloxyandrosta-1,4-diene-17β-carbothioate (11);
S-hydroxymethyl 6α,9α-difluoro-16α-methyl-3-oxo-11β-hydroxy-17α-propionyloxyandrosta-1,4-diene-17β-carbothioate (17);
S-hydroxymethyl 6α,9α-difluoro-11β,17α-dihydroxy-16α-methyl-3-oxo-androsta-1,4-diene-17β-carbothioate (21);
S-hydroxymethyl 6α,9α-difluoro-11β,16α,17α-trihydroxy-3-oxoandrosta-1,4-diene-17β-carbothioate16,17-acetonide (27); and
S-hydroxymethyl 9β,11β-epoxy-16α,17α-dihydroxy-3-oxoandrosta-1,4-diene-17β-carbothioate16,17-acetonide (33).

3. Process for the preparation of compounds of general formula (III) comprising the following steps:
d) reaction of aldehydes of formula R′″CHO, wherein R′″ is defined as above for the compound of formula (III), said aldehydes being possibly in the form of acetal, with a compound of general formula (II) 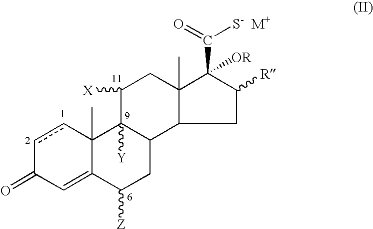
in which M⁺ is an ammonium or aminic ion, or M is H or an alkaline metal, to give a compound of general formula (III), said reaction being possibly carried out in the presence of strong mineral acids, when M is an alkaline metal or M+is an ammonium or aminic ion.

4. Process according to claim 3, wherein in the reaction of step d) the strong mineral acid, when present, is hydrochloric acid.

5. Process according to claim 3, wherein the said aldehyde in the reaction of step d) is formaldehyde.

6. Process according to claim 3, further comprising a reaction of selective fluorination of the hydroxylic group in alpha position with respect to the sulphur atom in compounds of general formula (III), reaction in step e) after step d), to give compounds of general formula (IV): 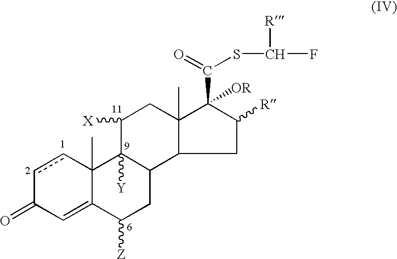
where R, R″, R′″, X, Y and Z have the same meaning as above, wherein the said reaction of selective fluorination is carried out with nucleophilic fluorination reagents.

7. Process according to claim 6, wherein the nucleophilic fluorination reagents are selected from the group consisting of bis(2-methoxyethyl) aminosulphur trifluoride, diethylamino sulphur trifluoride, and hexafluoropropyldiethylamine.

8. Process for the preparation of S-hydroxymethyl 6α-fluoro-9β,11β-epoxy-16α-methyl-3-oxo-17α-propionyloxyandrosta-1,4-diene-17β-carbothioate (5) according to claim 3, wherein the compound of general formula (II) is 6α-fluoro-9β,11β-epoxy-16α-methyl-17α-propionyloxy-3-oxoandrosta-1,4-diene-17β-thiocarboxylate of diethylammonium (4) which is made to react in step d) with formaldehyde to give S-hydroxymethyl 6α-fluoro-9β,11β-epoxy-16α-methyl-3-oxo-17α-propionyloxyandrosta-1,4-diene-17β-carbothioate (5).

9. Process according to claim 8, wherein the starting reagent, 6α-fluoro-9β,11β-epoxy-16α-methyl-17α-propionyloxy-3-oxoandrosta-1,4-diene-17β-thiocarboxylate of diethylammonium (4) is obtained by means of a process comprising the following steps:
a) reaction of 6α-fluoro-9β,11β-epoxy-17α-hydroxy-16α-methyl-3-oxoandrosta-1,4-diene-17β-carboxylic acid (1) with propionyl chloride in the presence of triethylamine to give 6α-fluoro-9β,11β-epoxy-16α-methyl-17α-propionyloxy-3-oxoandrosta-1,4-diene-170-carboxylic acid (2);
b) reaction of 6α-fluoro-9β,11β-epoxy-16α-methyl-17α-propionyloxy-3-oxoandrosta-1,4-diene-17β-carboxylic acid (2) coming from step a) with dimethylthiocarbamoyl chloride in the presence of sodium iodide and triethylamine to give 17β-N,N-dimethylthiocarbamoiloxycarbonyl-6α-fluoro-9β,11β-epoxy-16α-methyl-17α-propionyloxy-3-oxoandrosta-1,4-diene (3);
c) reaction of 17β-N,N-dimethylthiocarbamoiloxycarbonyl-6α-fluoro-9β,11β-epoxy-16α-methyl-17α-propionyloxy-3-oxoandrosta-1,4-diene (3) coming from step b) with diethylamine to give 6α-fluoro-9β,11β-epoxy-16α-methyl-17α-propionyloxy-3-oxoandrosta-1,4-diene-17β-thiocarboxylate of diethylammonium (4).

10. Process according to claim 8, further comprising a reaction of selective fluorination of the hydroxylic group in alpha position with respect to the sulphur atom of S-hydroxymethyl 6α-fluoro-9β,11β-epoxy-16α-methyl-3-oxo-17α-propionyloxyandrosta-1,4-diene-17β-carbothioate (5), reaction in step e) after step d), to give S-fluoromethyl 6α-fluoro-9β,11β-epoxy-16α-methyl-3-oxo-17α-propionyloxyandrosta-1,4-diene-17β-carbothioate (6), wherein the reaction of selective fluorination is carried out with nucleophilic fluorination reagents.

11. Process according to claim 10, wherein the nucleophilic fluorination reagents are selected from the group consisting of bis(2-methoxyethyl) aminosulphur trifluoride, diethylamino sulphur trifluoride, and hexafluoropropyldiethylamine.

12. Process for the preparation of S-hydroxymethyl 9β,11β-epoxy-3-oxo-17α-propionyloxyandrosta-1,4-diene-17β-carbothioate (11) according to claim 3, wherein the compound of general formula (II) is 9β,11β-epoxy-17α-propionyloxy-3-oxoandrosta-1,4-diene-17β-thiocarboxylate of diethylammonium (10) which is made to react in step d) with formaldehyde to give S-hydroxymethyl 9β,11β-epoxy-3-oxo-17α-propionyloxyandrosta-1,4-diene-17β-carbothioate (11).

13. Process according to claim 12, wherein the starting reagent, 9β,11β-epoxy-17α-propionyloxy-3-oxoandrosta-1,4-diene-17β-thiocarboxylate of diethylammonium (10) is obtained by means of a process comprising the following steps:
a) reaction of 9β,11β-epoxy-17α-hydroxy-3-oxoandrosta-1,4-diene-17β-carboxylic acid (7) with propionyl chloride in the presence of triethylamine to give 9β,11β-epoxy-17α-propionyloxy-3-oxoandrosta-1,4-diene-17β-carboxylic acid (8);
b) reaction of 9β,11β-epoxy-17α-propionyloxy-3-oxoandrosta-1,4-diene-17β-carboxylic acid (8) coming from step a) with dimethylthiocarbamoyl chloride in the presence of sodium iodide and triethylamine to give 170-N,N-dimethylthiocarbamoyloxycarbonyl-9β,11β-epoxy-17α-propionyloxy-3-oxoandrosta-1,4-diene (9);
c) reaction of 17β-N,N-dimethylthiocarbamoyloxycarbonyl-9β,11β-epoxy-17α-propionyloxy-3-oxoandrosta-1,4-diene (9) coming from step b) with diethylamine to give 9β,11β-epoxy-17α-propionyloxy-3-oxoandrosta-1,4-diene-17β-thiocarboxylate of diethylammonium (10).

14. Process according to claim 12, further comprising a reaction of selective fluorination of the hydroxylic group in position alpha with respect to the sulphur atom of S-hydroxymethyl 9β,11β-epoxy-3-oxo-17α-propionyloxyandrosta-1,4-diene-17β-carbothioate (11), reaction in step e) after step d), to give S-fluoromethyl 9β,11β-epoxy-3-oxo-17α-propionyloxyandrosta-1,4-diene-17β-carbothioate (12), wherein the reaction of selective fluorination is carried out with nucleophilic fluorination reagents.

15. Process according to claim 14, wherein the nucleophilic fluorination reagents are selected from the group consisting of bis(2-methoxyethyl) aminosulphur trifluoride, diethylamino sulphur trifluoride, and hexafluoropropyldiethylamine.

16. Process for the preparation of S-hydroxymethyl 6α,9α-difluoro-16α-methyl-3-oxo-11β-hydroxy-17α-propionyloxyandrosta-1,4-diene-17β-carbothioate (17) according to claim 3, wherein the compound of general formula (II) is 6α,9α-difluoro-16α-methyl-3-oxo-11β-hydroxy-17α-propionyloxyandrosta-1,4-diene-17β-thiocarboxylate of diethylammonium (16) which is made to react in step d) with formaldehyde to give S-hydroxymethyl 6α,9α-difluoro-16α-methyl-3-oxo-11β-hydroxy-17α-propionyloxyandrosta-1,4-diene-17β-carbothioate (17).

17. Process according to claim 16, wherein the starting reagent, 6α,9α-difluoro-16α-methyl-3-oxo-11β-hydroxy-17α-propionyloxyandrosta-1,4-diene-17β-thiocarboxylate of diethylammonium (16) is obtained by means of a process comprising the following steps:
a) reaction of 6α,9α-difluoro-11β,17α-dihydroxy-16α-methyl-3-oxoandrosta-1,4-diene-17β-carboxylic acid (13) with propionyl chloride in the presence of triethylamine to give 6α,9α-difluoro-16α-methyl-11β-hydroxy-17β-propionyloxy-3-oxoandrosta-1,4-diene-17β-carboxylic acid (14);
b) reaction of 6α,9α-difluoro-16α-methyl-11β-hydroxy-17α-propionyloxy-3-oxoandrosta-1,4-diene-17β-carboxylic acid (14) coming from step a) with dimethylthiocarbamoyl chloride in the presence of sodium iodide and triethylamine to give 17β-N,N-dimethylthiocarbamoyloxycarbonyl-6α,9α-difluoro-16α-methyl-3-oxo-11β-hydroxy-17α-propionyloxyandrosta-1,4-diene (15);
c) reaction of 17β-N,N-dimethylthiocarbamoyloxycarbonyl-6α,9α-difluoro-16α-methyl-3-oxo-11β-hydroxy-17α-propionyloxyandrosta-1,4-diene (15) coming from step b) with diethylamine to give 6α,9α-difluoro-16α-methyl-3-oxo-11β-hydroxy-17α-propionyloxyandrosta-1,4-diene-17β-thiocarboxylate of diethylammonium (16).

18. Process for the preparation of S-hydroxymethyl 6α,9α-difluoro-16α-methyl-3-oxo-11β-hydroxy-17α-propionyloxyandrosta-1,4-diene-17α-carbothioate (17) according to claim 3, wherein the compound of general formula (II) is 17β carbothioic 6α,9α-difluoro-16α-methyl-3-oxo-11β-hydroxy-17α-propionyloxyandrosta-1,4-diene acid (16a) which is made to react in step d) with formaldehyde to give S-hydroxymethyl 6α,9α-difluoro-16α-methyl-3-oxo-11β-hydroxy-17α-propionyloxyandrosta-1,4-diene-17β-carbothioate (17).

19. Process according to claim 18, wherein the starting reagent, 17β, carbothioic 6α,9α-difluoro-16α-methyl-3-oxo-11β-hydroxy-17α-propionyloxyandrosta-1,4-diene (16a) is obtained by means of a process comprising the following steps:
a) reaction of 6α,9α-difluoro-11β, 17α-dihydroxy-16α-methyl-3-oxoandrosta-1,4-diene-17β-carboxylic acid (13) with propionyl chloride in the presence of triethylamine to give 6α,9α-difluoro-16α-methyl-11β-hydroxy-17α-propionyloxy-3-oxoandrosta-1,4-diene-17β-carboxylic acid (14);
b) reaction of 6α,9α-difluoro-16α-methyl-11β-hydroxy-17α-propionyloxy-3-oxoandrosta-1,4-diene-17β-carboxylic acid (14) coming from step a) with dimethylthiocarbamoyl chloride in the presence of sodium iodide and triethylamine to give 17β-N,N-dimethylthiocarbamoyloxycarbonyl-6α,9α-difluoro-16α-methyl-3-oxo-11β-hydroxy-17α-propionyloxyandrosta-1,4-diene (15);
c′) reaction of 17β-N,N-dimethylthiocarbamoyloxycarbonyl-6α,9α-difluoro-16α-methyl-3-oxo-11β-hydroxy-17α-propionyloxyandrosta-1,4-diene (15) coming from step b) with sodium hydrogen sulphide followed by treatment with phosphoric acid to give 17β carbothioic 6α,9α-difluoro-16α-methyl-3-oxo-11β-hydroxy-17α-propionyloxyandrosta-1,4-diene acid (16a).

20. Process according to claim 16, further comprising after step d) a step e) of reaction of selective fluorination of the hydroxylic group in position alpha with respect to the sulphur atom of S-hydroxymethyl 6α,9α-difluoro-16α-methyl-3-oxo-11β-hydroxy-17α-propionyloxy-androsta-1,4-diene-17β-carbothioate (17), to give S-fluoromethyl 6α,9α-difluoro-16α-methyl-3-oxo-11β-hydroxy-17α-propionyloxyandrosta-1,4-diene-17β-carbothioate (18), wherein the reaction of selective fluorination is carried out with nucleophilic fluorination reagents.

21. Process according to claim 20, wherein the nucleophilic fluorination reagents are selected from the group consisting of bis(2-methoxyethyl) aminosulphur trifluoride, diethylamino sulphur trifluoride, and hexafluoropropyldiethylamine.

22. Process for the preparation of S-hydroxymethyl 6α,9α-difluoro-11β,17β-dihydroxy-16α-methyl-3-oxo-androsta-1,4-diene-17β-carbothioate (21) according to claim 3, wherein the compound of general formula (II) is 17β carbothioic 6α,9α-difluoro-11β,17β-dihydroxy-16α-methyl-3-oxo-androsta-1,4-diene acid (20) which is made to react in step d) with formaldehyde to give S-hydroxymethyl 6α,9α-difluoro-11β,17α-dihydroxy-16α-methyl-3-oxo-androsta-1,4-diene-17β-carbothioate (21).

23. Process according to claim 22 wherein the starting reagent, 17β carbothioic 6α,9α-difluoro-11β,17α-dihydroxy-16α-methyl-3-oxo-androsta-1,4-diene acid (20) is obtained by means of a process comprising the following steps:
b) reaction of 6α,9α-difluoro-11β,17α-dihydroxy-16α-methyl-3-oxoandrosta-1,4-diene-17β-carboxylic acid (13) with dimethylthiocarbamoyl chloride in the presence of sodium iodide and triethylamine to give 17β-N,N-dimethylthiocarbamoyloxycarbonyl-6α,9α-difluoro-11β,17α-dihydroxy-16α-methyl-3-oxoandrosta-1,4-diene (19);
c′) reaction of 17β-N,N-dimethylthiocarbamoiloxycarbonyl-6α,9α-difluoro-11β,17α-dihydroxy-16α-methyl-3-oxoandrosta-1,4-diene (19) coming from step b) with sodium hydrogen sulphide followed by treatment with phosphoric acid to give 17β carbothioic 6α,9α-difluoro-11β,17α-dihydroxy-16α-methyl-3-oxoandrosta-1,4-diene acid (20).

24. Process according to claim 22 further comprising after step d) a step e) of selective fluorination of the hydroxylic group in alpha position with respect to the sulphur atom of S-hydroxymethyl 6α,9α-difluoro-11β,17α-dihydroxy-16α-methyl-3-oxo-androsta-1,4-diene-17β-carbothioate (21), to give S-fluoromethyl 6α,9α-difluoro-11β,17α-dihydroxy-16α-methyl-3-oxo-androsta-1,4-diene-17β-carbothioate (22), wherein the reaction of selective fluorination is carried out with nucleophilic fluorination reagents.

25. Process according to claim 24 wherein the said nucleophilic fluorination reagents are selected from the group consisting of bis(2-methoxyethyl) aminosulphur trifluoride, diethylamino sulphur trifluoride, and hexafluoropropyldiethylamine.

26. Process for the preparation of S-hydroxymethyl 6α,9α-difluoro-11β,16α,17α-trihydroxy-3-oxoandrosta-1,4-diene-17β-carbothioate 16,17-acetonide (27) according to claim 3, wherein the compound of general formula (II) is 17β carbothioic 6α,9α-difluoro-11β,16α,17α-trihydroxy-3-oxoandrosta-1,4-diene 16,17-acetonide acid (26) which is made to react in step d) with formaldehyde to give S-hydroxymethyl 6α,9α-difluoro-11β,16α,17α-trihydroxy-3-oxoandrosta-1,4-diene-17β-carbothioate16,17-acetonide (27).

27. Process according to claim 26 wherein the starting reagent, 17β carbothioic 6α,9α-difluoro-11β,16α,17α-trihydroxy-3-oxoandrosta-1,4-diene 16,17-acetonide acid (26) is obtained by means of a process comprising the following steps:
a′) alkaline hydrolysis in the presence of air of 6α,9α-difluoro-11β, 16α,17α,21α-tetrahydroxy-1,4-pregnadiene-3,20-dione-16,17-acetonide-21 acetate (23) to give 6α,9α-difluoro-11β,16α,17α-trihydroxy-3-oxoandrosta-1,4-diene-17β-carboxylic 16,17-acetonide acid (24);
b) reaction of 6α,9α-difluoro-11β,16α, 17α-trihydroxy-3-oxoandrosta-1,4-diene-17β-carboxylic 16,17-acetonide acid (24) coming from step a′) with dimethylthiocarbamoyl chloride in the presence of sodium iodide and triethylaamine to give 17β-N,N-dimethylthiocarbamoyloxycarbonyl-6α,9α-difluoro-11β,16α, 17α-trihydroxy-3-oxoandrosta-1,4-diene 16,17-acetonide (25);
c′) reaction of 17β-N,N-dimethylthiocarbamoyloxycarbonyl-6α,9α-difluoro-11β,16α,17α-trihydroxy-3-oxoandrosta-1,4-diene 16,17-acetonide (25) coming from step b) with sodium hydrogen sulphide followed by treatment with phosphoric acid to give 17β carbothioic 6α,9α-difluoro-11β,16α,17α-trihydroxy-3-oxoandrosta-1,4-diene 16,17-acetonide acid (26).

28. Process according to claim 26 further comprising after step d) a step e) of selective fluorination of the hydroxylic group in position alpha with respect to the sulphur atom of S-hydroxymethyl 6α,9α-difluoro-11β,16α,17α-trihydroxy-3-oxoandrosta-1,4-diene-17β-carbothioate 16,17-acetonide (27), to give S-fluoromethyl 6α,9α-difluoro-11β,16α,17α-trihydroxy-3-oxoandrosta-1,4-diene-17β-carbothioate 16,17-acetonide (28), wherein the reaction of selective fluorination is carried out with nucleophilic fluorination reagents.

29. Process according to claim 28 wherein the nucleophilic fluorination reagents are selected from the group consisting of bis(2-methoxyethyl) aminosulphur trifluoride, diethylamino sulphur trifluoride, and hexafluoropropyldiethylamine.

30. Process for the preparation of S-hydroxymethyl 9β,11β-epoxy-16α,17α-dihydroxy-3-oxoandrosta-1,4-diene-17β-carbothioate 16,17-acetonide (33) according to claim 3, wherein the compound of general formula (II) is 17β carbothioic 9β,11β-epoxy-16α,17α-dihydroxy-3-oxoandrosta-1,4-diene 16,17-acetonide acid (32) which is made to react in step d) with formaldehyde to give S-hydroxymethyl 9β,11β-epoxy-16α,17α-dihydroxy-3-oxoandrosta-1,4-diene-17β-carbothioate16,17-acetonide (33).

31. Process according to claim 30 wherein the starting reagent, 17β carbothioic 9β,11β-epoxy-16α,17α-dihydroxy-3-oxoandrosta-1,4-diene 16,17-acetonide acid (32) is obtained by means of a process comprising the following steps:
a′) alkaline hydrolysis in the presence of air of 6α,9α-difluoro-9β,11β-epoxy-16α,17α,21-trhydroxy-1,4-pregnadiene-3,20-dione-16,17-acetonide-21 acetate (29) to give 9β,11β-epoxy-16α,17α-dihydroxy-3-oxoandrosta-1,4-diene-17β-carboxylic 16,17-acetonide acid (30);
b) reaction of 9β,11β-epoxy-16α,17α-dihydroxy-3-oxoandrosta-1,4-diene-17β-carboxylic 16,17-acetonide acid (30) coming from step a′) with dimethylthiocarbamoyl chloride in the presence of sodium iodide and triethylamine to give 17β-N,N-dimethylthiocarbamoyloxycarbonyl-9β,11β-epoxy-16α,17α-dihydroxy-3-oxoandrosta-1,4-diene 16,17-acetonide (31);
c′) reaction of 17β-N,N-dimethylthiocarbamoyloxycarbonyl-9β,11β-epoxy-16α,17α-dihydroxy-3-oxoandrosta-1,4-diene 16,17-acetonide (31) coming from step b) with sodium hydrogen sulphide followed by treatment with phosphoric acid to give 17β carbothioic 9β,11β-epoxy-16α,17α-dihydroxy-3-oxoandrosta-1,4-diene 16,17-acetonide acid (32).

32. Process according to claim 30 further comprising after step d) a step e) of selective fluorination of the hydroxylic group in alpha position with respect to the sulphur atom of S-hydroxymethyl 9β,11β-epoxy-16α,17α-dihydroxy-3-oxoandrosta-1,4-diene-17β-carbothioate 16,17-acetonide (33), to give S-fluoromethyl 9β,11β-epoxy-16α,17α-dihydroxy-3-oxoandrosta-1,4-diene-17β-carbothioate 16,17-acetonide (34), wherein the reaction of selective fluorination is carried out with nucleophilic fluorination reagents.

33. Process according to claim 32 wherein the said nucleophilic fluorination reagents are selected from the group consisting of bis(2-methoxyethyl) aminosulphur trifluoride, diethylamino sulphur trifluoride, and hexafluoropropyldiethylamine.

34. Process according to claim 10, further comprising a step f) of conversion of S-fluoromethyl 6α-fluoro-9β,11β-epoxy-16α-methyl-3-oxo-17α-propionyloxyandrosta-1,4-diene-17β-carbothioate (6) into S-fluoromethyl 6α,9α-difluoro-16α-methyl-3-oxo-11β-hydroxy-17α-propionyloxyandrosta-1,4-diene-17β-carbothioate (18) (fluticasone propionate) by reaction of S-fluoromethyl 6α-fluoro-9β,11β-epoxy-16α-methyl-3-oxo-17α-propionyloxyandrosta-1,4-diene-17β-carbothioate (6) with 70% hydrofluoric acid, at a temperature ranging from −30° C. to room temperature, to give S-fluoromethyl 6α,9α-difluoro-16α-methyl-3-oxo-11β-hydroxy-17α-propionyloxyandrosta-1,4-diene-17β-carbothioate (18).

35. Process according to claim 34 wherein the said reaction is carried out at a temperature ranging from −20° C. to 0° C.

36. Process according to claim 18, further comprising after step d) a step e) of reaction of selective fluorination of the hydroxylic group in position alpha with respect to the sulphur atom of S-hydroxymethyl 6α,9α-difluoro-16α-methyl-3-oxo-11β-hydroxy-17α-propionyloxy-androsta-1,4-diene-17β-carbothioate (17), to give S-fluoromethyl 6α,9α-difluoro-16α-methyl-3-oxo-11β-hydroxy-17α-propionyloxyandrosta-1,4-diene-17β-carbothioate (18), wherein the reaction of selective fluorination is carried out with nucleophilic fluorination reagents.

37. Process according to claim 21, wherein the nucleophilic fluorination reagents are selected from the group consisting of bis(2-methoxyethyl) aminosulphur trifluoride, diethylamino sulphur trifluoride, and hexafluoropropyldiethylamine.
